SEQUENCE LISTING (15 sequences)

SEQ ID NO: 1 - DNA, 339 bp, Artificial Sequence
  Chimeric Sequence (h5L)
gatattgtgatgacccagagcccgctgagcctgccggtgaccccaggcgaaccggcgtcgattagctgccgcagctcgcagaacatcgtgcataataacggcattacctatctggaatggtatctgcagaaaccgggccaaagcccgcagctgttaatttataaagtgagcgatcgctttagcggcgtgccggatcgcttttcgggcagcggtagtggcaccgattttacgctgaaaattagccgcgtggaagcggaggatgttggcgtgtattactgctttcagggcagccatatcccgccaacctttggccaaggcaccaaagtggaaattaaacgc

SEQ ID NO: 2 - protein, 113 aa, Artificial Sequence
  Synthetic Construct Chimeric Sequence (h5L)
DIVMTQSPLSLPVTPGEPASISCRSSQNIVHNNGITYLEWYLQKPGQSPQLLIYKVSDRFSGVPDRFSGSGSGTDFTLKISRVEAEDVGVYYCFQGSHIPPTFGQGTKVEIKR

SEQ ID NO: 3 - DNA, 354 bp, Artificial Sequence
  Chimeric Sequence (h5H)
caggtgcaactggttcagagcggcgcggaagtgaaaaagccgggcgcgtcggttaaagtgagctgcaaagcctcaggctatacctttacgagctactggatgcattgggtgcgccaggccccgggtcagggcctggaatggatgggtaacatttatccgggcagcggtggcaccaactatgcggaaaaatttaagaaccgcgtgaccatgacgcgtgataccagcatttcgacggcctatatggaactgagccgcctgcgtagcgatgacaccgccgtgtattactgcgcgcgcagtggcggtccgtattttttcgattactggggccagggtacgctggttaccgtgagctcg

SEQ ID NO: 4 - protein, 118 aa, Artificial Sequence
  Synthetic Construct Chimeric Sequence (h5H)
QVQLVQSGAEVKKPGASVKVSCKASGYTFTSYWMHWVRQAPGQGLEWMGNIYPGSGGTNYAEKFKNRVTMTRDTSISTAYMELSRLRSDDTAVYYCARSGGPYFFDYWGQGTLVTVSS

SEQ ID NO: 5 - DNA, 321 bp, Artificial Sequence
  Chimeric Sequence (hOL)
gatatccagatgacccagagcccgagctctctgagcgcgagcgtgggcgatcgcgtgaccattacgtgcagcgcgtctagctctgtgagctatatgaactggtaccagcaaaccccaggcaaagcgccgaaacgctggatttatgataccagcaaactggcgagcggcgtgccgagccgctttagcggctctggtagcggcaccgattatacgtttaccattagctctctgcagccggaagatattgcgacctattactgccagcaatggagctctaacccgtttacctttggccagggtaccaaactgcagattacccgc

SEQ ID NO: 6 - protein, 107 aa, Artificial Sequence
  Synthetic Construct Chimeric Sequence (hOL)
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 7 - DNA, 357 bp, Artificial Sequence
  Chimeric Sequence (hOH)
caggtgcaactggtgcagagcggcggtggcgttgtgcagccgggccgcagcctgcgcctgtcttgcaaagcgagcggctatacctttacgcgctataccatgcattgggtgcgccaggcgccgggcaaaggtctggaatggattggctatattaacccgtctcgcggctataccaactataatcagaaagtgaaagatcgctttaccattagccgcgataactctaaaaacaccgcgtttctgcagatggatagcctgcgcccggaagataccggcgtgtatttttgcgcgcgctactatgatgaccattatagcctggattattggggccagggcaccccggtgaccgttagctcg

SEQ ID NO: 8 - protein, 119 aa, Artificial Sequence
  Synthetic Construct Chimeric Sequence (hOH)
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYSLDYWGQGTPVTVSS

SEQ ID NO: 9 - DNA, 678 bp, Homo sapiens
  misc_feature: Fc region of human IgG2
tgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacgcctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 10 - DNA, 29 bp, Artificial Sequence
  back primer FNcoI-IgG2 hinge
nnnccatggtgttgtgtcgagtgcccacc

SEQ ID NO: 11 - DNA, 31 bp, Artificial Sequence
  forward primer FIgG2 Fc-SacII
nnnccgcggtcatttacccggagacagggag

SEQ ID NO: 12 - DNA, 44 bp, Artificial Sequence
  back primer FPreScission-IgG2 hinge
ctggaagttctgttccaggggccctgttgtgtcgagtgcccacc

SEQ ID NO: 13 - DNA, 33 bp, Artificial Sequence
  forward primer FCH3-XhoI
nnnctcgagtcatttacccggagacagggagag

SEQ ID NO: 14 - DNA, 32 bp, Artificial Sequence
  back primer FBamHI-h5L
nnnggatccgatattgtgatgacccagagccc

SEQ ID NO: 15 - DNA, 42 bp, Artificial Sequence
  forward primer FhOH-PreScission
gggcccctggaacagaacttccagggagctaacggtcaccgg

(19) United States
(12) Patent Application Publication
(10) Pub. No.: US 2017/0183410 A1
(43) Pub. Date: Jun. 29, 2017

(54) HIGHLY-FUNCTIONAL IgG2 BISPECIFIC ANTIBODY

(71) Applicant: TOHOKU UNIVERSITY, Sendai-shi, Miyagi (JP)

(72) Inventors: Izumi KUMAGAI, Sendai-shi (JP); Ryutaro ASANO, Sendai-shi (JP); Mitsuo UMETSU, Sendai-shi (JP)

(73) Assignee: TOHOKU UNIVERSITY, Sendai-shi, Miyagi (JP)

(21) Appl. No.: 15/128,831

(22) PCT Filed: Feb. 25, 2015
(86) PCT No.: PCT/JP2015/055341

§ 371 Date: Mar. 6, 2017

(30) Foreign Application Priority Data
Mar. 25, 2014 (JP) 2014-062066

(51) Int. Cl. C07K 16/28; C07K 16/46

(52) U.S. Cl. CPC C07K 16/2863; C07K 16/2809; C07K 16/468; C07K 2317/31; C07K 2317/24; C07K 2317/56; C07K 2317/622; C07K 2317/14; C07K 2317/92; C07K 2317/94; A61K 2039/505

(57) ABSTRACT

To provide a high-functional bispecific antibody, especially LH-type bispecific antibody that has further improved its function while maintaining the excellent cytotoxicity such as anti-tumor activity. A bispecific antibody for human EGF receptor 1 and CD3, comprising a variable region comprising a humanized variable region of the light chain (5L: SEQ ID NO:2) and a humanized variable region of the heavy chain (5H: SEQ ID NO:4) of an anti-human EGF receptor 1 antibody 528, and a humanized variable region of the light chain (OL: SEQ ID NO:6) and of a humanized variable region of the heavy chain (OH: SEQ ID NO:8) of an anti-CD3 antibody OKT; a hinge region; and an Fc region, characterized by that the Fc region is originated from human IgG2 sub-class.

### FIELD OF THE INVENTION

The present invention is related to a humanized highly functional IgG2 bispecific antibody, which is superior in stability and can be used in a cancer-specific immunotherapy, a single-chain polypeptide constituting the antibody, a nucleic acid encoding the polypeptide, a method for the production of the antibody, use of them as a pharmaceutical preparation, etc.

### BACKGROUND OF THE INVENTION

Recently, immunotherapy has been used as a safe therapy for the treatment of cancer, rheumatoid, etc. An antibody showing a cancer-specific cytotoxic activity (cytotoxicity) is used in the immunotherapy of cancer. While it is recognized that an antibody drug comprising such antibody will show high and safe therapeutic effects with little side effects, it has a problem that it would cost much since said drug needs to be produced by using established animal cells.

As a result, the production of recombinant antibodies having an extremely strong activity has been tried as a means for reducing a dosage so as to attain low cost.

Among these recombinant antibodies, an antibody with bispecificity (Bispecific Antibody: BsAb) has been studied intensively. This is because the bispecific antibody can bind specifically to two different kinds of antigens so that it will be utilized as a therapeutic agent having a specific anti-cancer effect. A diabody (Db) is a minimum unit of the above bispecific antibody. It was developed by utilizing the property that the variable region in a heavy chain (VH) and the variable region in a light chain (VL) originated from the same parent antibody will form a hetero-dimer through non-covalent bond (Non-Patent Document 1). Methods for the production of bispecific antibodies other than the diabody-type bispecific antibody are described in Non-Patent Documents 2 and 3.

The present inventors already found that the diabody-type bispecific antibody (Ex3) that was produced by utilizing an anti-human EGF receptor 1 (Her 1) antibody 528 and an anti-CD3 antibody OKT3, and its humanized diabody-type bispecific antibody (referred to as “hEx3” in Patent Document 1) showed extremely strong anti-tumor effects. Furthermore, the present inventors have developed a highly functional bispecific antibody having various structures, based on said humanized diabody-type bispecific antibody (Patent Document 2).

The present inventors have also developed a LH-type bispecific antibody that is characterized in that the variable region of a light chain is located at an N-end (N-terminal) side of each polypeptide constituting the humanized diabody-type bispecific antibody, and a humanized highly functional bispecific antibody comprising said LH-type bispecific antibody (Patent Document 3). They have further developed antibodies wherein the heavy or light chain of the Her 1 antibody 528 has various kinds of mutation/substitution of amino acid(s) (Patent Documents 4 and 5).

The highly functional bispecific antibodies disclosed in Patent Documents 2-5 are bispecific antibodies that comprise an Fc region in addition to the variable region containing the light and heavy chains of the anti-human EGF receptor 1 antibody 528 and the anti-CD3 antibody OKT3. These humanized highly functional bispecific antibodies have a significantly increased cytotoxicity when compared with Ex3, and a divalent binding activity for each antigen. A bispecific antibody with a minimized additional sequence such as Tag may be easily prepared by digestion with a protease, and easily purified with Protein A. They are further provided with an effector property such as induction of an antibody-dependent cellular cytotoxicity (ADCC) activity and a complement-dependent cytotoxicity (CDC) function.

### RELATED ARTS

### Patent Document

Patent Document 1: Japanese Patent No. 3803790

Patent Document 2: WO 2007/108152 A1

Patent Document 3: WO 2010/109924 A1

Patent Document 4: WO 2011/062112 A1

Patent Document 5: WO 2012/020622 A1

### Non-Patent Document

Non-Patent Document 1: Hollinger, et al., Proc. Natl. Acad. Sci. USA 90, 6444-6448, 1993

Non-Patent Document 2: Alt M, et. al. Novel tetravalent and bispecific IgG-like antibody molecules combining single-chain diabodies with the immunoglobulin gammal Fc or CH3 region. FEBS Lett., 454, 90-4. (1999)

Non-Patent Document 3: Lu D, et. al. A fully human recombinant IgG-like bispecific antibody to both the epidermal growth factor receptor and the insulin-like growth factor receptor for enhanced antitumor activity. J Biol Chem., 280, 19665-72. (2005)

Non-Patent Document 4: J Biol Chem, 2005:280 (20) 19665-72

### DISCLOSURE OF THE INVENTION

### Problem to be Solved by the Invention

Although the bispecific antibodies (BsAb) comprising the Fc region have extremely excellent properties, they have a problem that fragmentation shall occur in the vicinity of a hinge region in a fusion site between the variable region and Fc region during a storing period after their purification (FIG. 1). There are also concerns about side effects that are caused by the over-induction of the effector property via the Fc region.

Especially, although the LH-type highly functional bispecific antibody disclosed in Patent Document 3 (LH-type Ex3-scDb-Fc) shows superior activity in vitro to its HL-type antibody, no significant difference is observed in vivo such as in tumor established models. Accordingly, a further improvement in the function has been desired with respect to the above LH-type bispecific antibodies.

The problem to be solved by the present invention is therefore to provide a clinically more excellent bispecific antibody that has improved the above problems while maintaining the excellent cytotoxicity such as anti-tumor activity.

### Means for Solving the Problems

The Fc region originated from IgG1 sub-class is usually used in the art in the production of the bispecific antibodies comprising an Fc region so as to effectively induce the effector property (Non-Patent Document 4). However, the present inventors have focused on the fact that IgG2 sub-class has a low inducing activity of the effector property among the sub-classes of the Fc region, and tried the production of a highly functional bispecific antibody wherein the IgG1-originated Fc region conventionally used is replaced by the IgG2-originated Fc region, leading to the accomplishment of the present invention that has solved the above problems.

The present invention is therefore related to the following aspects:

[1] A bispecific antibody for human EGF receptor 1 and CD3, comprising a variable region comprising a humanized variable region of the light chain (5L: SEQ ID NO:2) and a humanized variable region of the heavy chain (5H: SEQ ID NO:4) of an anti-human EGF receptor 1 antibody 528, and a humanized variable region of the light chain (OL: SEQ ID NO:6) and of a humanized variable region of the heavy chain (OH: SEQ ID NO:8) of an anti-CD3 antibody OKT; a hinge region; and an Fc region, characterized by that the Fc region is originated from human IgG2 sub-class.

[2] The antibody according to the aspect [1], wherein the variable region of the light chain is located at an N-end side of the variable region of the heavy chain (LH-type) in each polypeptide.

[3] The antibody according to the aspect [1] or [2], which has the same domain number as an IgG-type immunoglobulin.

[4] The antibody according to the aspect [3], wherein a single polypeptide chain having the structure represented by (OL5H)-(peptide linker)-(5LOH) is bonded to the Fc region of a human antibody via a hinge region.

[5] A single-chain polypeptide constituting the antibody of any one of the aspects [1]-[4].

[6] A nucleic acid molecule encoding the polypeptide of the aspect [5].

[7] A replicable cloning vector or an expression vector containing the nucleic acid molecule of the aspect [6].

[8] The vector of the aspect [7], which is a plasmid vector.

[9] A host cell transformed with the vector of the aspect [7] or [8].

[10] The hose cell of the aspect [9], which is a mammalian cell.

[11] A method for the production of the antibody of any one of the aspects [1]-[4], comprising culturing a host cell according to the aspect [9] to express the nucleic acid molecule, and collecting and purifying the single-chain polypeptides according to the aspect [5], assembling the resulting single-chain polypeptides, and separating and collecting the antibody thus formed.

[12] A pharmaceutical composition comprising the antibody of any one of the aspects [1]-[4] as an active ingredient.

[13] The pharmaceutical composition of the aspect [12] for use in eliminating, hurting, damaging and/or reducing tumor cells.

### Advantages of the Invention

Although there was no difference in the antigen-binding activity between the antibody according to the present invention and the conventional same-type antibody comprising the Fc region originated from IgG1, the growth capability for PBMC was decreased in the antibody according to the present invention.

It was found that the antibody according to the present invention had a cytotoxic activity comparable to the conventional one where T-LAK cells were used as an effector cell. However, the antibody according to the present invention surprisingly showed a higher cytotoxic activity than the conventional one where PBMC was used as the effector cell, which was a different result from that expected based on the decrease in a PBMC-growth capability.

An unexpected result was also obtained with respect to a T cell-activating capability, that the capability of the antibody according to the present invention was lower for T-LAK, but higher for PBMC than that of the conventional same-type antibody.

Furthermore, the antibody according to the present invention showed a higher tumor-growth inhibiting effects in a vivo test than the conventional same-type antibody.

As one of the reasons, it is thought that since PBMC comprises Fc receptor (FcR)-positive NK cells showing the effector property, the function of the conventional same-type antibody having the FcR-binding capability to lymphocytes will be inhibited due to the steric hindrance caused by its binding to the NK cells under co-existence of the lymphocytes and the NK cells in PBMC

The same reason is conceived for the vivo test since the mice used therein possess the NK cells showing the cross reaction with the human Fc region.

Further, the antibody according to the present invention shows an excellent stability when compared to the conventional same-type antibody that has the Fc region originated from IgG1.

### BRIEF DESCRIPTION OF THE DRAWINGS

FIG. 1 shows one example of fragmentation of the LH-type Ex3-scDb-Fc.

FIG. 2 shows the construction of an expression vector of LH-type IgG2 of Ex3-scDb-Fc.

FIG. 3 shows the results of confirmation of the expression by FCM.

FIG. 4 shows the results of cloning (480 clones) with ELISA.

FIG. 5 shows the results of cloning (12 clones) with ELISA.

FIG. 6 shows the results of purification with Protein A.

FIG. 7 shows the results of purification with gel-filtration chromatography.

FIG. 8 shows the results of Proliferation assay using PBMC.

FIG. 9 shows the results of evaluation of cytotoxicity by MTS assay.

FIG. 10 shows the results of evaluation of capability of inducing cytokine secretion (only cancer cells+T-LK).

FIG. 11 shows the results of evaluation of inducing capability of cytokine secretion (only T-LK).

FIG. 12 shows amino acid sequences of the hinge regions of IgG1 and IgG2.

FIG. 13 shows the results of Proliferation assay using T-LAK.

FIG. 14 shows the results of evaluation of induction of T cell activation by FCM.

FIG. 15 shows the results of evaluation of stability of LH-type IgG2 of Ex3-scDb-Fc.

FIG. 16 shows the results of in vivo test of LH-type IgG2 of Ex3-scDb-Fc.

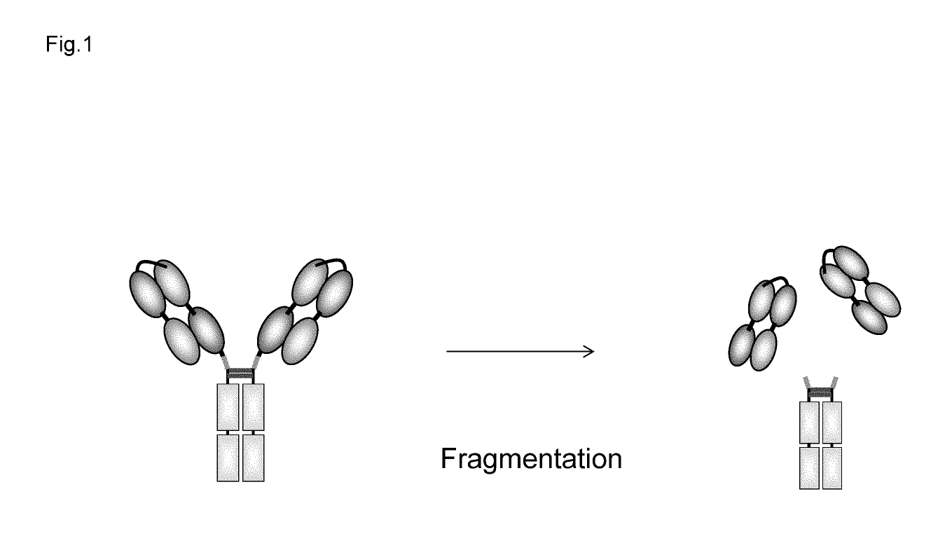

### BEST MODE FOR CARRYING OUT THE INVENTION

The antibody according to the present invention comprises the variable region comprising the humanized variable region of the light chain (5L: SEQ ID NO:2) and the humanized variable region of the heavy chain (5H: SEQ ID NO:4) of the anti-human EGF receptor 1 antibody 528, and the humanized variable region of the light chain (OL: SEQ ID NO:6) and of the humanized variable region of the heavy chain (OH: SEQ ID NO:8) of the anti-CD3 antibody OKT; the hinge region; and the Fc region originated from human IgG2 sub-class. As a result, the antibody according to the present invention possesses bi-specificity for human EGF receptor 1 and CD3. The term “Fc region” means a region comprising two domains (CH2 and CH3) constituting a constant region (C region), which is located at C-end side of the heavy chain; and the hinge region.

As the antibody according to the present invention comprises the human Fc region, it is easily purified with Protein A, and can induce the antibody-dependent cellular cytotoxicity (ADCC) and the complement-dependent cytotoxicity (CDC) function.

Furthermore, since the antibody according to the present invention uses the polypeptide originated from the human IgG2 sub-class as the Fc region, it shows higher stability and cytotoxicity than the conventional same-type bispecific antibody that has the Fc region originated from IgG1.

Various types of the antibodies may be produced as an example of the bispecific antibody comprising the above constituents, which may include, for example, the highly functional bispecific antibodies with a conventional IgG-type antibody molecule such as the second type (Ex3-Fc) and the third type (Ex3 scDb-Fc) disclosed in Patent Document 2; and the highly functional bispecific antibody that is described as the fourth type (Ex3 scFv-Fc) comprising a light chain constant region (CL) and a heavy chain constant region (CH1) in addition to the above constituents.

There is no limitation in the light chain constant region (CL) and the heavy chain constant region (CH1) comprised in the bispecific antibody of the fourth type mentioned above as long as they are originated from the human antibody. For example, CL may be originated from κ or λ chain. The CH1 is usually originated from γ chain of IgG. Examples of the CH1 and CL are those having an amino acid sequence represented by SEQ ID NO:29 and SEQ ID NO:33, respectively, in Patent Document 2.

More specifically, the second type (Ex3-Fc) has the structure wherein the humanized diabody-type bispecific antibody (Ex3) consisting of the two kinds of the polypeptides of (OH5L) and (5H OL) is bonded to the two Fc regions of the human antibody via each hinge region through either of the two polypeptides. Thus, this is composed of one of the two kinds of the polypeptides constituting Ex3, which has been bonded to the Fc region of the human antibody via each hinge region (for example, (5HOL)-(hinge region)-Fc region), and the other polypeptide (for example, OH5L). The above antibody may be produced by expressing the two kinds of the polypeptides and assembling them.

In the antibody of the above type, either 5HOL or OH5L may be bonded to the Fc region of the human antibody via the hinge region, and either the heavy or light chain variable region may be bonded to the hinge region.

The third type (Ex3 scDb-Fc) has the structure wherein a single-chain polypeptide of (OH5L)-(a peptide linker)-(5HOL), (OH5H)-(a peptide linker)-(5LOL) or a tandem-type (5L5H)-(a peptide linker)-(OHOL) may be bonded to the Fc region of the human antibody via each hinge region. Any one of the two kinds of the heavy chains or light chains in the single-chain polypeptide may be bonded to the hinge region.

As the number of the domains constituting the second and third type of the present antibody is the same as that of an immunoglobulin molecule of the IgG type, it is considered that these antibodies have a steric structure similar to that of the immunoglobulin molecule. A protease cleavage site may be inserted between the hinge region and Ex3 or Ex3 scDb in the second or third type of the present BsAb. As a result, Ex3 or Ex3 scDb can be easily produced by digesting these BsAb with the protease followed by the purification steps mentioned below. The Ex3 or Ex3 scDb thus produced by the protease digestion will show stronger cytotoxic activity than those produced by the conventional methods.

The fourth type (Ex3 scFv-Fc) has the structure wherein the VH and VL of the human antibody are replaced by the single-chain Fv (scFv) (5HL) comprising the humanized variable regions of the heavy chain (5H) and the light chain (5L) of the anti-human EGF receptor 1 antibody 528, and the single-chain Fv (OHL) comprising the humanized variable regions of the heavy chain (OH) and the light chain (OL) of an anti-CD3 antibody OKT3, respectively, or vice versa. Thus, this BsAb is an IgG-type immunoglobulin composed of two polypeptides, i.e., a polypeptide wherein one of the scFv of OHL and 5HL is bonded to the N-end of CH1 domain constituting the constant region of the heavy chain, and a polypeptide wherein the other scFv is bonded to the N-end of CL domain constituting the constant region of the light chain. And, either the heavy or light chain variable region in each scFv may be bonded to the constant region. The above antibody may be produced by expressing the two kinds of the single-chain polypeptides and assembling them.

The antibodies according to the present invention further comprise various antibodies wherein the variable region of the light chain is located at the N-end (N-terminal) side of the variable region of the heavy chain in each polypeptide constituting the bispecific antibody (LH-type). Thus, an example of such LH-type of the third type antibody (Ex3 scDb-Fc) has the structure wherein the single polypeptide having the structure of (OLSH)-(a peptide linker)-(SLOH) is bonded to the Fc region of the human antibody via the hinge region, as described in an example of the present specification.

Various amino acid mutation and/or replacement may be inserted into the heavy or light chain of the anti-Her 1 antibody 528 in each polypeptide that constitutes the bispecific antibody according to the present antibody, as shown in Patent Document 4 or 5.

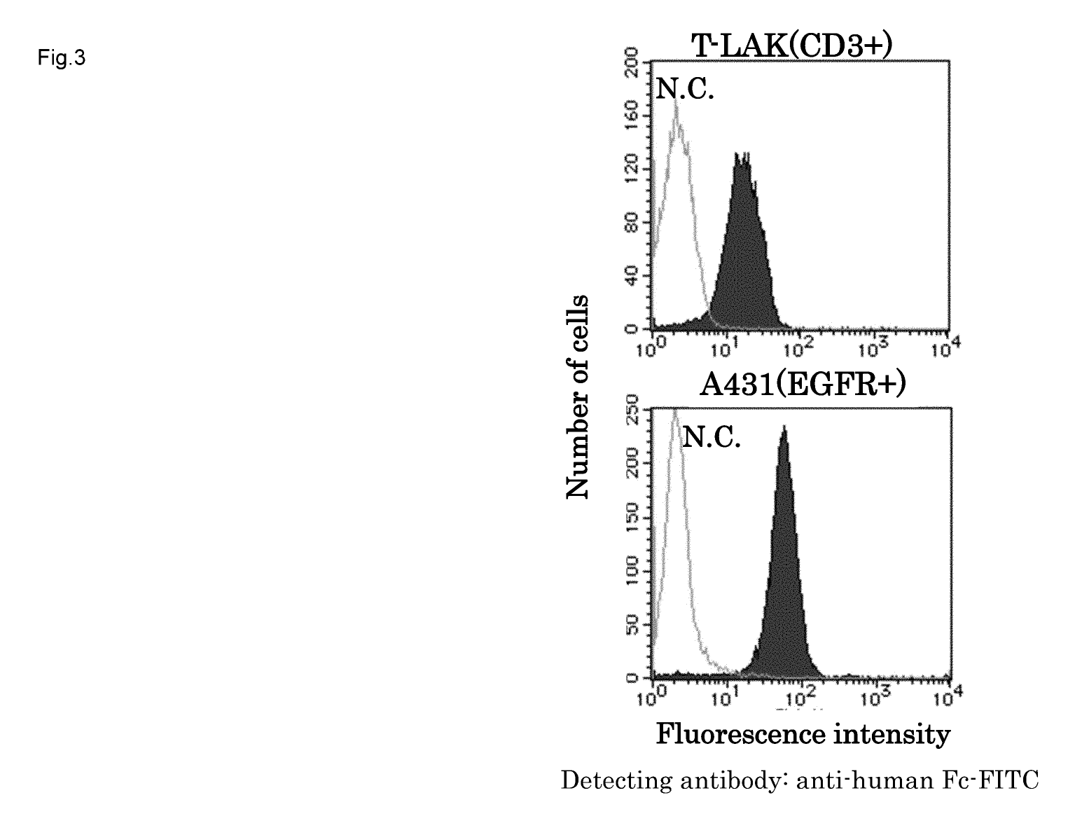

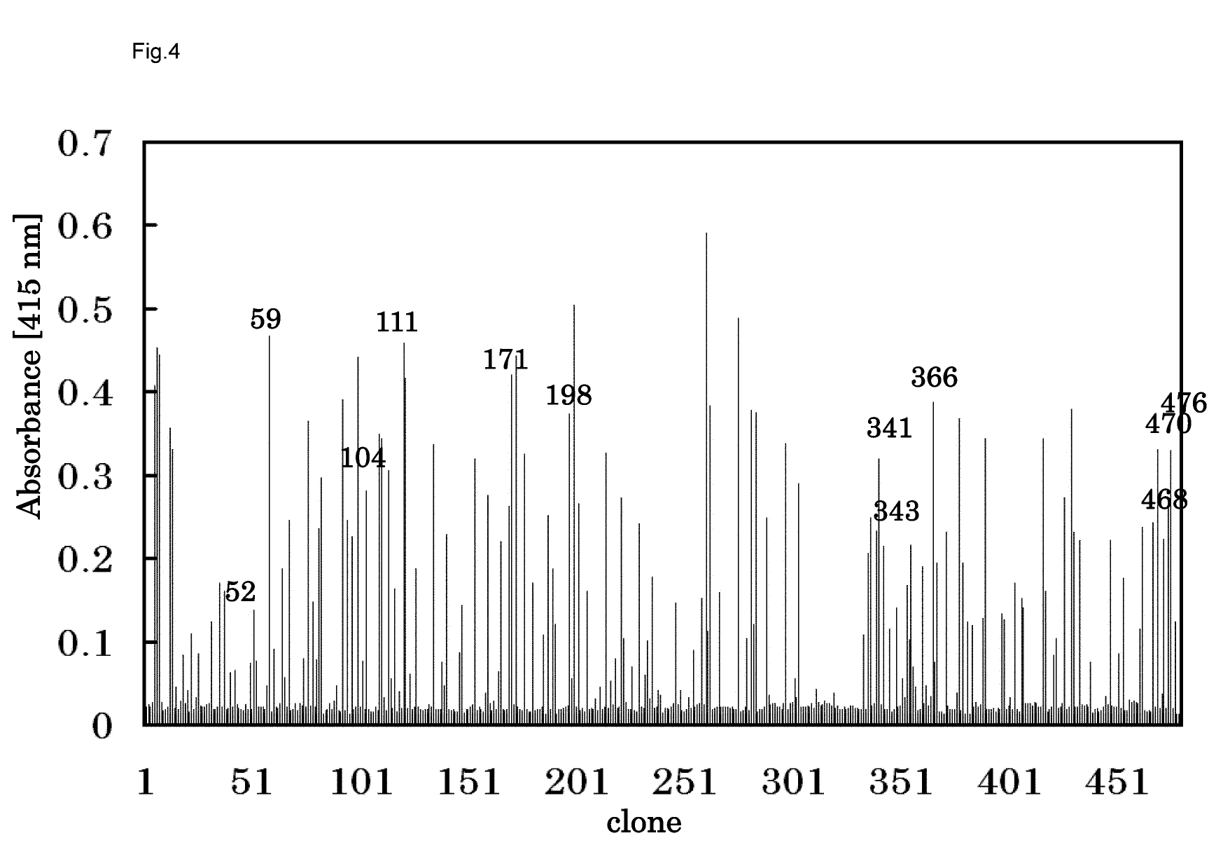

The antibody according to the present invention may comprises amino acid sequences of the PreSission sequence, peptide linker, signal peptide, etc., as shown in FIGS. 3-3 and 3-4 of Patent Document 2. The PreSission sequence comprises a protease-cleavage site. There is no limitation on the kind of protease used in the present invention, and any enzyme known in the art such as Thrombin and Factor Xa may be used, and the amino acid sequence comprising the protease-cleavage site may be optionally selected accordingly.

It is, however, preferable not to comprise the protease-cleavage site in the PreSission sequence so as to more effectively inhibit the fragmentation of the present antibody.

Mouse B cell hybridoma 528 producing anti-EGFR antibody (ID:TKG0555) is deposited in Cell Resource Center for Biomedical Research, Institute of Development, Aging and Cancer, TOHOKU University. The above hybridoma 528 producing anti-EGFR antibody is also stored at ATCC with an ATCC Accession No. HB-8509, so that may be obtained from these deposit authorities.

On the other hand, a hybridoma producing the anti-CD3 antibody, OKT3 (ID:TKG0235), is deposited in Cell Resource Center for Biomedical Research, Institute of Development, Aging and Cancer, TOHOKU University, and is also stored at ATCC with an ATCC Accession No. CRL-8001, so that it may be obtained from these deposit authorities.

cDNA may be prepared by known methods using these hybridomas. For example, mRNA is extracted with ISOGEN (Nippon Gene Co.) and then cDNA is prepared by means of First-Strand cDNA Synthesis Kit (Amersham Biosciences Co.). PCR reaction is done for the cDNA using cloning primers that are synthesized in accordance with the disclosure of a Reference document (Krebber, A. et al. Reliable cloning of functional antibody variable domains from hybridomas and spleen cell repertoires employing a re-engineered phage display system. J Immunol Methods 201, 35-55. (1997)) so as to determine the sequences of the variable regions of H and L chains of each antibody.

The term “humanized” variable region comprised in the single-polypeptide constituting the bispecific antibody according to the present invention means a human immunoglobulin (a recipient antibody) in which at least a part of the residues of complementary-determining region (CDR) is replaced with residues derived from the CDR of a non-human animal antibody (a donor antibody) that has a desired specificity, affinity and capability, such as those of mouse, rat, and rabbit. In some cases, the residue(s) of a Fv framework (FR) in the human immunoglobulin is replaced with residue(s) of the corresponding non-human antibody. The humanized antibody may further comprise a residue that is not found in the recipient antibody or the introduced CDR or framework. These changes are made in order to optimize or improve the properties of the resulting antibody. More detailed information on these changes are referred to Jones et al., Nature 321, 522-525 (1986); Reichmann et al., Nature 332, 323-329 (1988); EP-B-239400; Presta, Curr. Op. Struct. Biol 2, 593-596 (1992); and EP-B-451216.

The humanized variable region of the antibody may be prepared in accordance with any methods known to those skilled in the art, for example, by analyzing various conceptual humanized preparations based on three-dimensional immunoglobulin models of the recipient antibody and donor antibody, and analyzing them. The three-dimensional immunoglobulin models are well known in the art, being referred to, for example, WO 92/22653.

Thus, one example of the humanized variable region according to the present invention is an antibody wherein the complementary determining regions (CDR) in the variable regions are originated from a mouse antibody, and the other parts are originated from a human antibody.

The activity or function of the resulting antibody may be deteriorated due to the humanization. The activity or function of the bispecific antibody according to the present invention may be therefore improved by being provided with a site-specific mutation at an appropriate position in the single-chain polypeptide, for example, at a position in the framework which can affect the CDR structure, such as in canonical sequence or vernier sequence.

Specifically, the humanization of the variable regions of 528 was performed by means of CDR grafting. Thus, a human antibody having FR (Frame Work) with the highest homology was screened and selected by a homology search in view of the length of each CDR and the like. An amino acid sequence was designed, in which the CDR of the selected human antibody was replaced with the CDR of 528. The total gene may be then synthesized by means of overlapping PCR by preferably using the optimum codons for the host cell used in the expression.

It was already reported that the variable region of the humanized OKT3 could sufficiently maintain its activity when compared with the mouse OKT3 (Adair, J. R. et al. Humanization of the murine anti-human CD3 monoclonal antibody OKT3. Hum Antibodies Hybridomas 5, 41-7. (1994)). The total gene was synthesized by means of overlapping PCR based on the amino acid sequence of the variable regions of the humanized OKT3 disclosed in the above document. The optimum codons for the host cell were preferably used in the synthesis. It was also reported that the use of the gene containing the optimum codons would increase the expression level in the host cell.

In addition to the peptide linkers shown in each of the above single-chain polypeptide, it is preferred that the humanized variable regions of the light chain (VL) and the heavy chain (VH) are linked via an appropriate peptide linker. Any linker known in the art or one modified therefrom may be optionally selected and used in the present invention, as long as it makes hard for the single-chain polypeptide to interact within its molecule so that it will enable the formation of a polymer made of plurality of the single-chain antibodies. As a result, the VH and VL comprised in the different single-chain antibodies shall assemble appropriately with each other so as to form a structure that mimics or improves the function of an original protein (the above polypeptide was originated or derived from the original protein) such as all or part of its biological activity. The peptide linker according to the present invention may have 1-20 amino acids, preferably 1-15 amino acids, more preferably 2-10 amino acids.

Alternatively, the two humanized variable regions may be directly linked with each other in the single-chain polypeptide. In such case, one or a few amino acids located at the C-end of the humanized variable region of N-end side fragment, or one or a few amino acids located at the N-end of the humanized variable region of C-end side fragment are deleted in order to increase three-dimensional degree of freedom in each single-chain antibody and to improve their polymerization.

The polypeptide having an amino acid sequence in which one or a few amino acids such as, for example, 2-5 amino acids are substituted, deleted, inserted or added in the amino acid sequences represented by the above SEQ ID NOS, and having substantially the same property and function as that of the original polypeptide such as an antigen specificity of its variable region may be also used as the single chain polypeptide constituting the present BsAb. It is preferable to make a substitution among amino acids belonging to the same group (polar, non-polar, hydrophobic, hydrophilic, positive-charged, negative-charged, or aromatic amino acid group), or to make a deletion or addition of amino acid so as not to cause a substantial difference or effects with respect to the three-dimensional or local charge-condition of the protein. Such polypeptides having the substitution, deletion or addition of the amino acid(s) my be easily prepared by well known methods such as site-specific mutation (point mutation method or cassette mutation), genetic homologous recombination, primer extension method and PCR, or any optional combinations thereof. The above amino acid sequences comprising one or few amino acids that are substituted, deleted, inserted or added have homology (identity) of 90% or more, preferably 95% or more, more preferably 99% or more with a full-length amino acid sequence of the original amino acid sequence.

The representative examples of the nucleic acid molecules (oligonucleotides) encoding the whole or part of the amino acid sequences of the single-chain polypeptide according to the present invention have the nucleotide sequences shown in the above SEQ ID NOS. Furthermore, as a nucleic acid molecule with the nucleotide sequence having homology of 90% or more, preferably 95% or more, more preferably 99% or more with a full-length nucleotide sequence represented by the above SEQ ID NOS is considered to encode a polypeptide having substantially the same property and function as that of the original polypeptide or part thereof, the above nucleic acid molecule is included in the nucleic acid molecule of the present invention. Although the nucleic acid molecule comprises a nucleotide sequence encoding at least either of the two kinds of the single-chain polypeptides constituting the BsAb according to the present invention, it preferably comprises two kinds of nucleotide sequences together, each one of which encodes one of the two kinds of said single-chain polypeptides, respectively.

In order to determine the homology between two amino acid or nucleotide sequences, they may be preliminarily treated into an optimum condition for comparison. For example, a gap may be inserted into one of the sequences to optimize the alignment with the other sequence, followed by the comparison of amino acid or nucleotide at each site. When the same amino acid or nucleotide exists at a corresponding site of the first and second sequences, these two sequences are considered to be identical with respect to said site. Homology between two sequences is shown by a percent ratio of the number of the identical sites over the total number of amino acids or nucleotides between the two sequences.

The term “homology (identity)” in this specification means an amount (or a number) of the amino acids in an amino acid sequence or the nucleotides in a nucleotide sequence, which are determined to be identical with each other in the relationship between two sequences, showing an extent of the correlation between the two polypeptide or nucleotide sequences. The homology may be easily calculated. The term “homology” or “identity” is well known in the art, and many methods for the calculation of such homology are known, among them. For example, Lesk, A. M. (Ed.), Computational Molecular Biology, Oxford University Press, New York, (1988); Smith, D. W. (Ed.), Biocomputing: Informatics and Genome Projects, Academic Press, New York, (1993); Grifin, A. M. & Grifin, H. G. (Ed.), Computer Analysis of Sequence Data: Part I, Human Press, New Jersey, (1994);von Heinje, G., Sequence Analysis in Molecular Biology, Academic Press, New York, (1987); Gribskov, M. & Devereux, J. (Ed.), Sequence Analysis Primer, M-Stockton Press, New York, (1991). A general method for the determination of the homology between two sequences is disclosed, for example, in Martin, J. Bishop (Ed.), Guide to Huge Computers, Academic Press, San Diego, (1994); Carillo, H. & Lipman, D., SIAM J. Applied Math., 48: 1073 (1988). A preferable method for the determination of the homology between two sequences is, for example, one designed to obtain a largely related part between said two sequences. Some of them are provided as a computer program. Although preferable examples of the computer programs for the determination of the homology between two sequences include, but not limited to, GCG program package (Devereux, J. et al., Nucleic Acids Research, 12(1): 387 (1984)), BLASTP, BLASTN, FASTA (Atschul, S. F. et al., J. Molec. Biol., 215:403 (1990), any method known in the art may be used.

The nucleic acid of the present invention further includes a DNA molecule that hybridizes with a DNA comprising a nucleotide sequence complementary to the nucleotide sequence represented by the above SEQ ID NOS under stringent conditions, and encodes a polypeptide having substantially the same property and function as that of the polypeptides represented by the above SEQ ID NOS.

Hybridization may be carried out by or in accordance with a method well known in the art such as that described in Molecular cloning third. ed. (cold Spring Harbor Lab. Press, 2001). Hybridization may be done in accordance with an instruction or manual attached to a commercially available library.

Hybridization may be carried out by or in accordance with a method well known in the art such as that described in Current protocols in molecular biology edited by Frederick M. Ausbel et al., 1987). Hybridization may be done in accordance with an instruction or manual attached to a commercially available library.

The phrase “stringent conditions” in this specification may be defined by a suitable combination of salt concentration, organic solvent (for example, formamide), temperature, and other known conditions. Thus, stringency will be increased by the decrease of salt concentration, or the increase of an organic solvent concentration or hybridization temperature. The washing conditions after the hybridization may also affect the stringency. The washing conditions are also defined by salt concentration and temperature. The stringency of washing will be increased by the decrease of salt concentration or the increase of temperature.

Accordingly, the “stringent conditions” in this specification means conditions under which a specific hybrid can be formed only between the nucleotide sequences having homology of about 80% or more, preferably about 90% or more, more preferably about 99% or more on a total average. Specifically, they may be sodium concentration of 150-900 mM, preferably 600-900 mM, pH6-8 at 60-68° C. One example of the stringent conditions is hybridization in 5×SSC (750 mM NaCl, 75 mM Na₃ Citrate), 1% SDS, 5×Denhart solution 50% formaldehyde at 42° C., followed by the washing with 0.1×SSC (15 mM NaCl, 1.5 mM Na₃ Citirate), 0.1% SDS at 55° C.

Furthermore, the nucleic acid encoding the humanized variable regions in the single-chain polypeptide of the present invention may be synthesized by means of the over-lapping PCR method based on a pre-determined amino acid sequence. The nucleic acid used herein has no limitation in its chemical structure or preparation route, as long as it is a molecule encoding the single-chain polypeptide, including gDNA, cDNA chemically-synthesized DNA and mRNA.

Specifically, the nucleic acid according to the present invention may be isolated from cDNA library by means of hybridization or PCR based on the sequences disclosed in literatures. The thus isolated DNA may be inserted in an expression vector, with which a host cell such E. coli, COS cell, CHO cell or myeloma not expressing immunoglobulin are transfected to synthesize a monoclonal antibody in the thus transformed host cells. PCR may be carried out in accordance with a method known in the art, or substantially the same or altered methods. The methods disclosed in, for example, R. Saiki, et al., Science, 230:1350, 1985; R. Saiki, et al., Science, 239:487, 1988; H. A. Erlich ed., PCR Technology, Stockton Press, 1989; D. M. Glover et al., ed., “DNA Cloning,” 2^{nd}. ed., Vol.1, (The Practical Approach Series), IRL Press, Oxford University Press (1995); M. A. Innis et al., ed., “PCR Protocols: a guide to methods and applications,” Academic Press, New York (1990); M. J. McPherson, P. Quirke and G. R. Taylor (Ed.), PCR: a practical approach, IRL Press, Oxford (1991); M. A. Frohman e al., Proc. Natl. Acad. Sci. USA, 85, 8998-9002 (1988), and their modified and altered methods may be used in the present invention. PCR may be performed with use of a commercially available kit in accordance with manufacturer's protocols.

The sequencing method of nucleic acids such as DNA may be referred to Sanger et al., Proc. Natl. Acad. Sci. USA 74:5463-5467 (1977). A general method for recombinant DNA techniques may be referred to J. Sambrook, E. F. Fritsch & T. Maniatis (ed.), “Molecular Cloning: A Laboratory Manual (2^{nd} edition)”, Cold Spring Harbor Laboratory Press, Cold Spring Harbor, N.Y. (1989) and D. M. Glover et al. (ed.), 2^{nd} ed., Vol. 1 to 4 (The Practical Approach Series), IRL Press, Oxford University Press (1995).

The nucleic acid encoding the single-chain polypeptide constituting the present BsAb or each region contained therein may be modified or altered so that it will optionally encode a desired peptide or amino acid depending on the purpose. The techniques for such modification or alternation are disclosed in Mutagenesis: a Practical Approach, M. J. McPherson (ed.), IRL Press, Oxford, UK (1991), including a site-specific mutagenesis introduction method, cassette mutagenesis induction method and PCR mutagenesis method.

The term “modification (or alternation)” as used herein refers to insertion, deletion or substitution of base(s) in at least one codon encoding an amino acid residue in the originally obtained nucleic acid. It includes alternation of the amino acid sequence per se of the single-chain polypeptide by replacing a codon encoding the original amino acid with a codon encoding another amino acid.

Alternatively, the nucleic acid encoding the single-chain polypeptide may be altered without changing the amino acid per se, by using a codon suitable for the host cell such as CHO cell (an optimum codon). With the use of the optimum codon, expression efficiency of the single-chain polypeptide in the host cell will be improved.

The antibody according to the present invention may be produced by various methods well known in the art such as genetic engineering technique and chemical synthesis. For example, the genetic engineering technique includes producing a replicable cloning vector or an expression vector containing the nucleic acid molecule encoding each of the two kinds of the single-chain polypeptides constituting the above bispecific antibody, transforming a host cell with the vector, culturing the transformed host cell to express each of the single-chain polypeptides, collecting and purifying said single-chain polypeptides, assembling the two kinds of the single-chain polypeptides, and separating and collecting the bispecific antibody thus formed.

The term “replicable expression vector” or “expression vector” as used herein refers to a piece of DNA (usually double-stranded) that may comprise a fragment of a foreign DNA fragment inserted therein. The foreign DNA is also defined as a “heterologous DNA”, which can not be found naturally in a host cell in interest. The vector is used to carry or convey the foreign or heterologous DNA into an appropriate host cell. Once the vector is introduced into the host cell, it may be replicated independently from a chromosomal DNA of the host cell to produce copies of the vector and foreign DNA inserted therein. The vector also comprises elements essential for translating the foreign DNA into a polypeptide so that the polypeptide molecules encoded by the foreign DNA will be synthesized very quickly.

The above vector means a DNA construct comprising an appropriate control sequence and DNA sequence that are operably linked together (i.e., linked together so that the foreign DNA can be expressed). The control sequence includes a promoter for transcription, an optional operator sequence to regulate the transcription, a sequence encoding an appropriate mRNA ribosome-biding site, an enhancer, a polyadenylation sequence, and a sequence controlling the termination of transcription and translation. The vector may further comprise various sequences known in the art, such as a restriction enzyme cleaving site, a marker gene (selection gene) such as a drug-resistant gene, a signal sequence, and a leader sequence. These sequences and elements may be optionally selected by those skilled in the art depending on the kinds of the foreign DNA and host cell, and conditions of culture medium. Furthermore, various peptide tags (c-myc and His-tag, for example) known in the art may be contained at its end, etc.

The vector may be in any form such as a plasmid, phage particle, or just simply genomic insert. Once the appropriate host cell is transformed with the vector, the vector will be replicated or function independently from the genome of the host cell, or the vector will alternatively be integrated into the genome of the cell.

The various expression vectors that are used in the production of the single polypeptide constituting the antibody according to the present invention may be easily constructed by those skilled in the art using the techniques known in the art.

Examples of the above vectors are described in Patent Document 1, especially in Examples 1, 2, 11 and 12, and examples of Patent Document 2.

Any cell known in the art may be used as the host cell, for example, there may be mentioned prokaryotic cells such as including E. coli., eukaryotic cells such as mammalian cells such Chinese hamster ovary (CHO) cell and human cells, yeast, and insect cells. For example, BL21 star (DE3) strain is cultured in 2×YT culture medium at about 28° C. and induced with IPTG of about 0.5 mM, so that the yield of the present LK-type bispecific antibody may be highly improved so as to increase its production efficiency.

Although the single-chain polypeptide obtained by the expression in the host cell is usually secreted and collected from the culture medium, it may be also collected from cell lysate when it is directly expressed without a secretion signal. In case the single-chain polypeptide has a membrane-binding property, it may be released from the membrane with an appropriate surfactant such as Triton-X100.

Purification of the polypeptide may be carried out by any method known to those skilled in the art such as centrifugation, hydroxyapatite chromatography, gel electrophoresis, dialysis, separation on ion-exchange chromatography, ethanol precipitation, reverse phase HPLC, silica chromatography, heparin-sepharose chromatography, anion- or cation-resin chromatography such as polyaspartic acid column, chromato-focusing, SDS-PAGE, precipitation with ammonium sulfate, and affinity chromatography. The affinity chromatography, which utilizes affinity with a peptide tag of the single-chain polypeptide, is one of the preferred purification techniques with a high efficiency.

Since the collected single-chain polypeptide may be often included in an insoluble fraction, the polypeptide is preferably purified after being solubilized and denatured. The solubilization treatment may be carried out with the use of any agent known in the art, including alcohol such ethanol, a dissolving agent such as guanidine hydrochloride and urea. The present BsAb is produced by assembling or rewinding the two kinds of the single-chain polypeptides thus purified, and separating and collecting the thus formed antibody molecule.

Assembling treatment will bring a single-chain polypeptide back in its appropriate spatial arrangement in which a desired biological activity is shown. Since this treatment may also bring polypeptides or domains back into their assembling state, it may be considered “re-assembling.” It may be also called “re-constitution” or “refolding” in view of gaining the desired biological activity. The assembling treatment may be carried out by any method known in the art, preferably by gradually lowering the concentration of a denaturing agent such as guanidine hydrochloride in a solution comprising the single-chain polypeptide by means of dialysis. During these processes, an anti-coagulant or oxidizing agent may be optionally added in a reaction system in order to promote the oxidation. The separation and collection of the present highly functional BsAb thus formed may be done by any method known in the art as well.

As already described above, the BsAb according to the present invention may be prepared from the supernatant of a culture medium, periplasm fraction, intracellular soluble fraction and intracellular insoluble fraction.

It is possible to transform the host cell with the co-expression vector containing the nucleic acid molecule encoding each of the single-chain polypeptides constituting the antibody of the present invention, or with the two kinds of the expression vector containing the nucleic acid molecule encoding each of said single-chain polypeptides, respectively, culturing the transformed host cell so as to express each of the single-chain polypeptides, allowing the transformed cell to form the antibody in said cell, and separating and collecting it from supernatant of the culture medium or intracellular soluble fraction. In such case, the above assembling or rewinding treatment is unnecessary so that a high productivity can be achieved at a low cost.

A pharmaceutical composition according to the present invention comprises an active ingredient selected from the group consisting of the antibody according to the present invention, the single-chain polypeptide, the nucleic acid, the vector, and the host cell described in the above. As shown by the examples in the present specification, since the active ingredient has an activity of eliminating, hurting, damaging and/or reducing tumor cells expressing EGFR in vitro and in vivo, the present pharmaceutical composition is used as an anti-tumor agent.

An effective amount of the active ingredient may be optionally determined by those skilled in the art depending on the purpose of treatment, medical conditions of a patient to be treated such as kind, site or size of tumor, and administration route. A typical dose or daily dose may be first determined in vitro by using an assay method of growth or existence of the tumors known in the art, then determined with use of such an appropriate animal model as to allow extrapolation of the resulting dose range to human patients.

The pharmaceutical composition of the present invention may optionally comprise various kinds of pharmaceutically acceptable components known in the art such as carrier, excipient, buffer, stabilizing agent and the like, depending on various factors such as the kind of the active ingredients, its formulation form, the route and purpose of administration, medical conditions of patient.

The pharmaceutical composition of the present invention may be formulated into any form such as pill, liquid, powder, gel, air spray, microcapsule, and colloidal dispersion (liposome, micro emulsion, etc.).

The pharmaceutical preparation may be administered by injecting or infusing intravenously, intraperitoneally, intracerebrally, intraspinally, intramuscularly, intraocularly, intraarterially, especially intrabiriarily, or via diseased tissue, or with use of a constant releasing agent system. The active ingredient according to the present invention may be administered through continuous fluid infusion or massive injection. The pharmaceutical composition according to the present invention is preferably administered in combination with the cell having phagocytosis or cytotoxic activity. Alternatively, the active ingredient such as the present BsAb may be mixed with the above cells so as to bind to them before its administration.

The constant releasing agent generally refers to a formulation that can release the active ingredient of the present invention for a certain period of time. One of the preferred constant releasing agents comprises a semi-permeable carrier of solid hydrophobic polymer such as protein, which is shaped into a form such as film or micro capsule.

The pharmaceutical preparation according to the present invention may be produced by a method that is optionally selected from, for example, “Guide Book of Japanese Pharmacopoeia”, Ed. of Editorial Committee of Japanese Pharmacopoeia, Version No.13, published Jul. 10, 1996 by Hirokawa publishing company

The terms as used in the present specification and drawings are based on IUPAC-IUB Commission on Biochemical Nomenclature or on meanings of the terms conventionally used in the art.

The present invention will be explained more in detail by referring to the Examples, which are provided only for describing the specific embodiments of the present invention, but not for limiting the scope of the present invention. It is therefore to be understood that various embodiments based on the inventive concept of the present specification may be practiced within the scope of the present invention.

The following examples were or can be carried out with standard techniques well known to those skilled in the art unless otherwise described. Thus, unless otherwise described, specific procedures and treating conditions are in accordance with J. Sambrook, E. F. Fritsch & T. Maniatis, “Molecular Cloning”, 2nd ed., Cold Spring Harbor Laboratory, Cold Spring Harbor, N.Y. (1989) and D. M. Glover et al. ed., “DNA Cloning”, 2nd ed., Vol. 1 to 4, (The Practical Approach Series), IRL Press, Oxford University Press (1995) (DNA cloning), and with H. A. Erlich ed., PCR Technology, Stockton Press, 1989 ; D. M. Glover et al. ed., “DNA Cloning”, 2nd ed., Vol. 1, (The Practical Approach Series), IRL Press, Oxford University Press (1995) and M. A. Innis et al. ed., “PCR Protocols”, Academic Press, New York (1990) (PCR). A commercially available agent and kit were used in accordance with protocols attached thereto.

The effects of the binding capability to a FcR or complement on cytotoxicity were examined by producing the LH-type IgG2 of Ex3-scDb-Fc wherein the hinge and Fc region of the conventional IgG1 in the LH-type Ex3-scDb-Fc had been changed with those of IgG2 that were considered to have the lower binding capability to the FcR or complement.

### EXAMPLE 1

Production of LH-Type IgG2 of Ex3-scDb-Fc

First, mRNA was extracted from PBMC with a method known to those skilled in the art, and cDNA was synthesized. A gene was amplified with PCR using primers that covered from the hinge region to Fc region of the human IgG2 sequence, and inserted into a pRA vector to give pRA IgG2 hinge-Fc. It was confirmed that a sequence of the resulting gene was completely identical with the human igG2 gene sequence (BX640623.1) in GenBank.

The resulting base sequence comprising the hinge and Fc regions (CH2 and CH3) of the human IgG2 sequence is represented by SEQ ID NO:9.

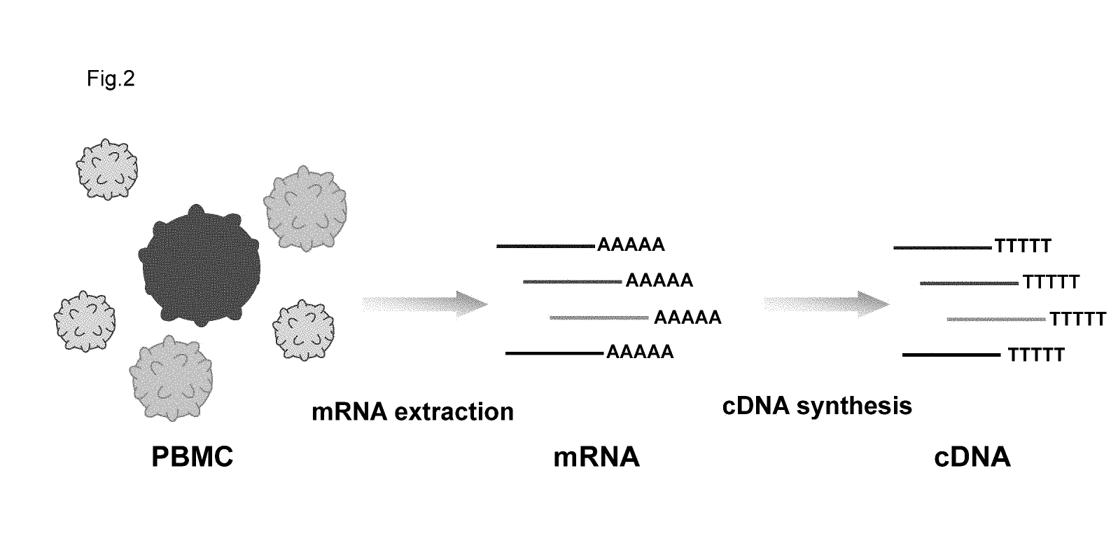

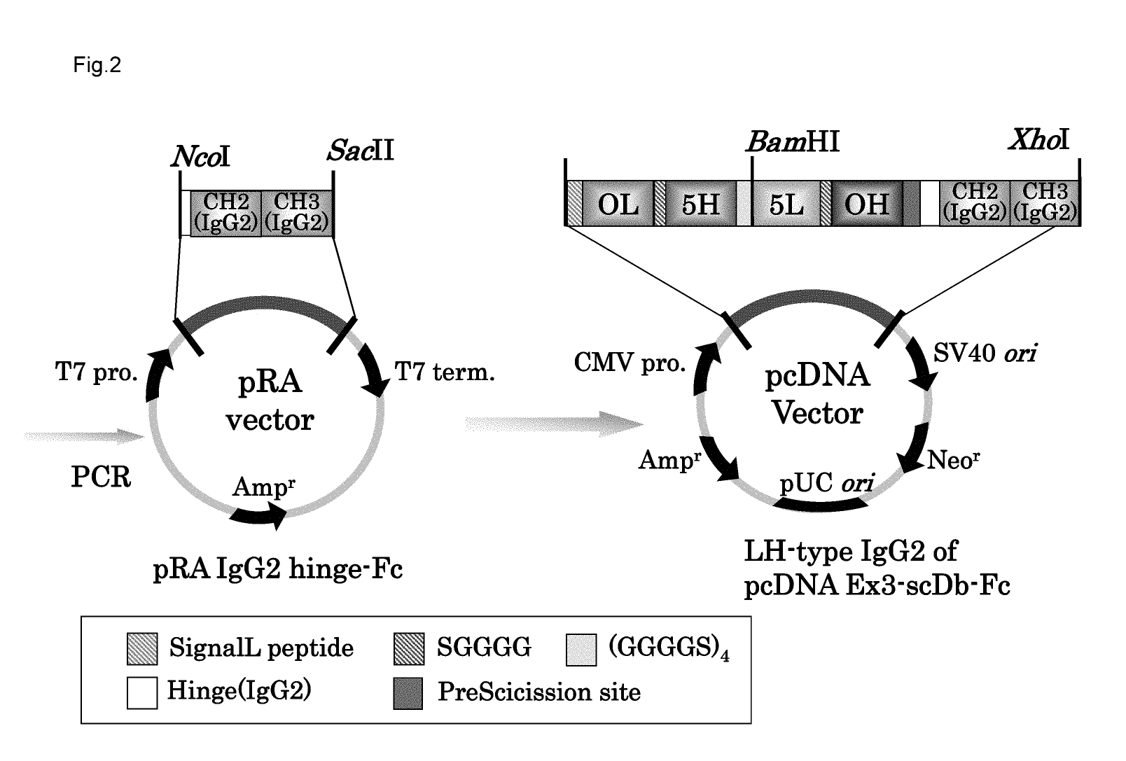

The second PCR was carried out using the resulting pRA IgG2 hinge-Fc and pcDNA LH-type Ex3-scDb-Fc produced in accordance with Example 2 of Patent Document 3 as a template, followed by digestion with BamHI and Xhol and ligation to give pcDNA LH-type IgG2 of Ex3-scDb-Fc (FIG. 2). The primers and conditions used are as follows.

pRA IgG2 hinge-Fc
(template: mRNA extract, annealing temperature:
55° C., elongation time: 60 sec)
back primer: NcoI-IgG2 hinge
SEQ ID NO: 10:
5′-NNNCCATGGTGTTGTGTCGAGTGCCCACC-3′
forward primer: IgG2 Fc-SacII
SEQ ID NO: 11:
5′-NNN CCGCGG TCATTTACCCGGAGACAGGGAG-3′
pcDNA LH-type IgG2 of Ex3-scDb-Fc
1^{st} PCR (production of PreScission
site-IgG2 Fc-XhoI)
(template: pRA IgG2 hinge-Fc, annealing
temperature: 55° C., elongation time: 60 sec)
back primer: PreScission-IgG2 hinge
SEQ ID NO: 12:
5′-CTGGAAGTTCTGTTCCAGGGGCCC TGTTGTGTCGAGTGCC
CACC-3′
forward primer: CH3-XhoI
SEQ ID NO: 13:
5′-NNNCTCGAGTCATTTACCCGGAGACAGGGAGAG-3′
1^{st} PCR (production of BamHI-5LOH-PreScission site)
(template: pcDNA LH-type Ex3-scDb-Fc, annealing
temperature: 60° C., elongation time: 60 sec)
back primer: BamHI-h5L
SEQ ID NO: 14:
5′-NNNGGATCC GATATTGTGATGACCCAGAGCCC-3′
forward primer: hOH-PreScission
SEQ ID NO: 15:
5′-GGGCCCCTGGAACAGAACTTCCAG GGAGCTAACGGTCACCGG-3′
2^{nd} PCR
(template: 1^{st} PCR product, annealing temperature:
60° C., elongation time: 60 sec)
back primer: BamHI-h5L
SEQ ID NO: 14
forward primer: CH3-XhoI
SEQ ID NO: 13

TABLE 1
Preparation of reaction solution

| | |
|---|---|
| Template DNA | 1 μL |
| Primer (10 pmol/μL) | 1 μL each |
| 10 × PCR buffer for KOD+ | 10 μL |
| dNTP 2 mM each | 10 μL |
| 25 mM MgSO₄ | 4 μL |
| KOD+ | 1 μL |
| ddH₂O | 72 μL |
| Total | 100 μL |

### EXAMPLE 2

Introduction and Cloning of the Gene into CHO Cell

The expression vector of pcDNA LH-type IgG2 of Ex3-scDb-Fc was digested with a restriction enzyme of Nrul to be linearized. The gene was then introduced into a host CHO cell with a liposome method using Lipofectamine 2000. After screening in a selective medium comprising G418, the binding activity to a human epidermoid cancer cell, A431 (ATCC No. CRL-1555) and a cytotoxic T cell, T-LAK (CD3+) was evaluated using a flow cytometry (FCM) in accordance with a method known for those skilled in the art, for example, the method in Example 2 of Patent Document 5 so as to confirm the expression of LH-type IgG2 of Ex3-scDb-Fc, which is one of the antibodies according to the present invention (FIG. 3). The introduction of the gene was confirmed since the binding activity was observed both to A431 and T-LAK.

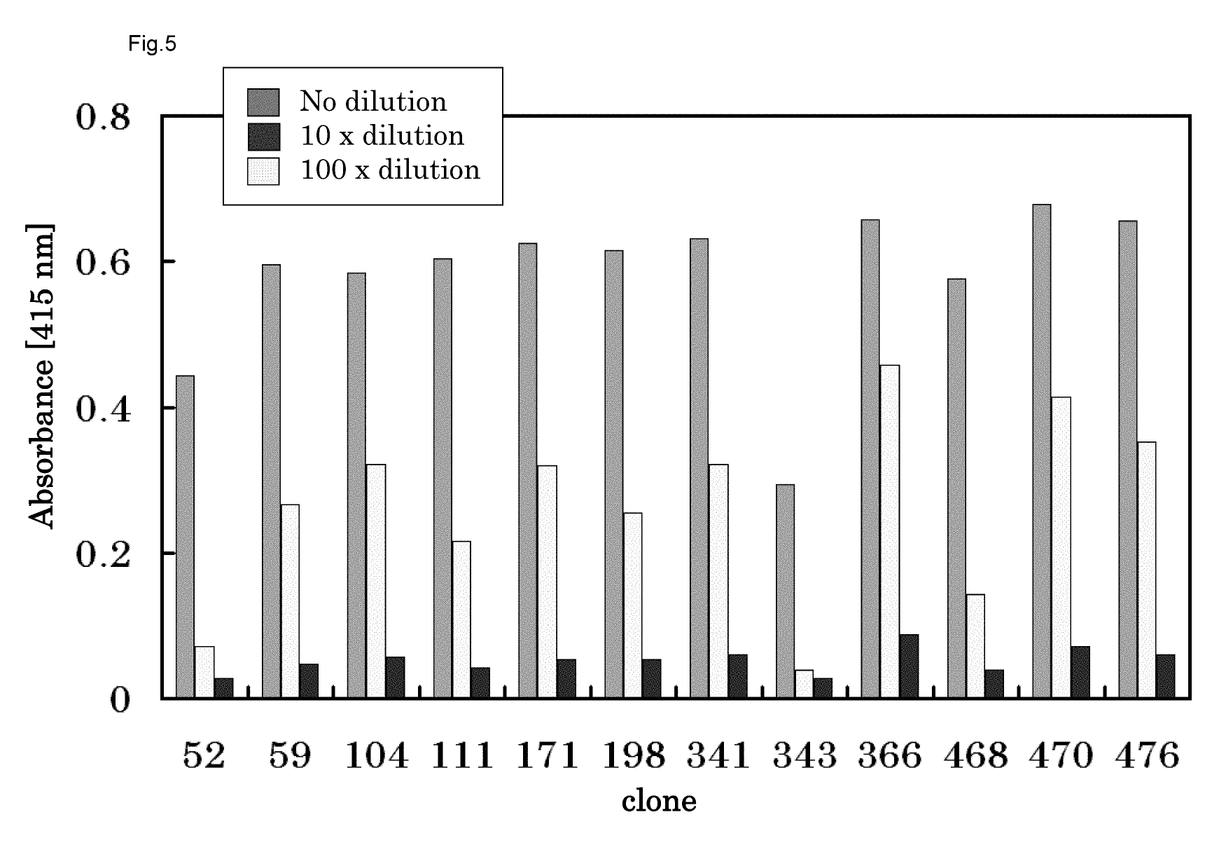

Cloning was carried out with a limiting dilution, followed by ELISA using their supernatant (FIG. 4). Clones 52, 59, 104, 111, 171, 198, 341, 343, 366, 468, 470 and 476 were selected, which formed a colony from only one cell and showed an especially high value, and subjected to an expanded culture in a 12-well plate. Their supernatant was diluted 10 times and 100 times to be used again in ELISA to select clones 366 and 470 that showed a higher value than the other clones in both their original solution and 10 times dilution (FIG. 5). ELISA was carried out using Human IgG ELISA Quantitation Kit (BETHYL Corporation).

Preparation of LH-Type IgG2 of Ex3-scDb-Fc

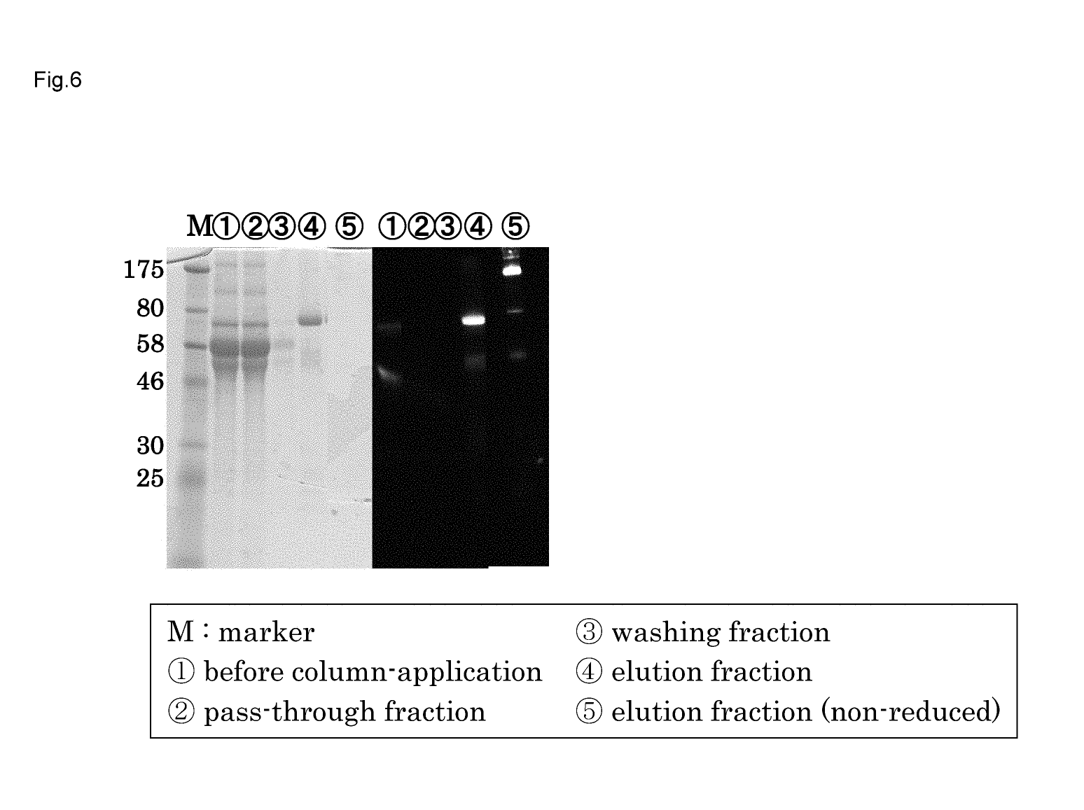

The resulting clones 366 and 470 were cultured in a triple flask, respectively, and their supernatant was subjected to purification with protein A. The purification was confirmed as a band in an elution fraction was observed in SDS-PAGE and western blotting (FIG. 6). Since the yield after the purification of clone 366 and clone 470 was 0.6 mg/mL and 0.9 mg/mL, respectively, the above confirmation and the following assay were carried out using clone 470.

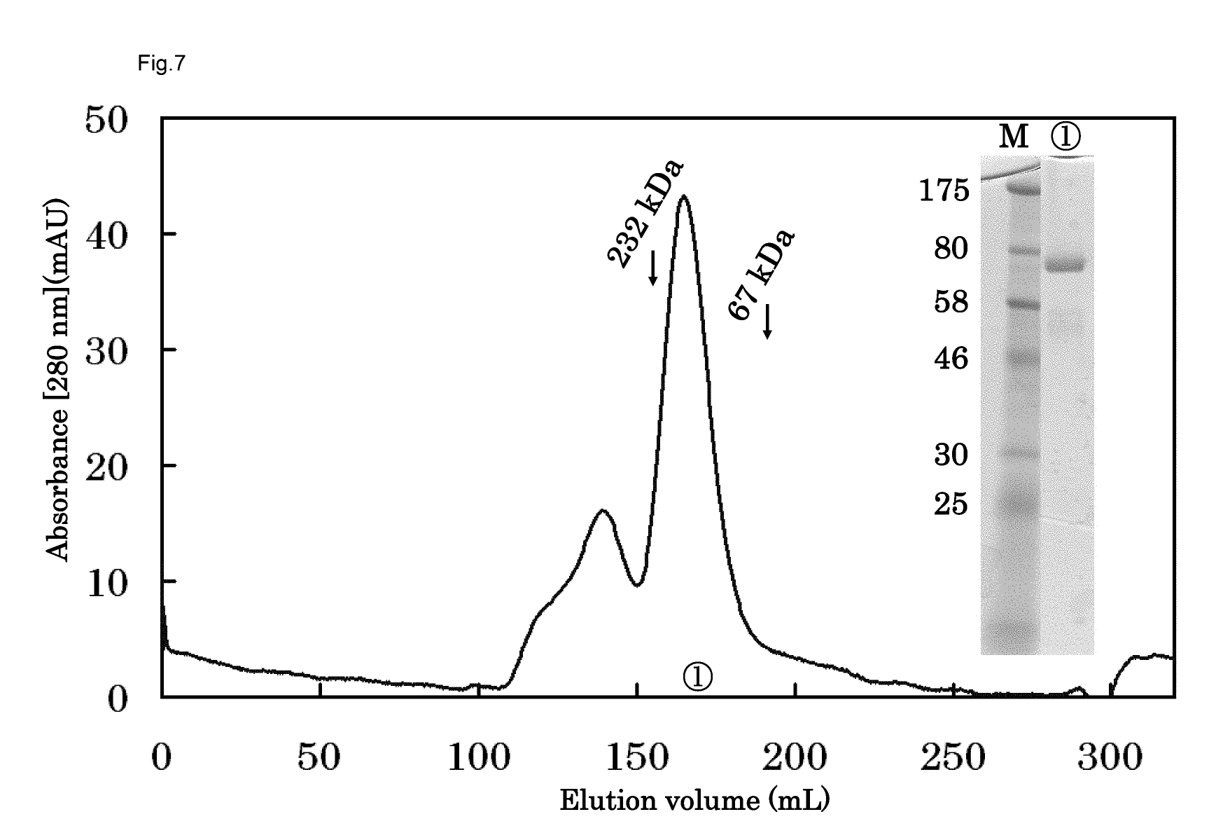

The final purification was carried out with a gel filtration chromatography of the elution fraction obtained in the above protein A purification. As a result, since a peak was observed in an elution fraction corresponding to the molecular weight of LH-type IgG2 of Ex3-scDb-Fc (158 kDa) (FIG. 7), the fraction was collected and subjected to the following assays.

### EXAMPLE 3

Evaluation of the Function of LH-Type IgG2 of Ex3-scDb-Fc

(1) PBMC Growth-Inducing Capability by Proliferation Assay

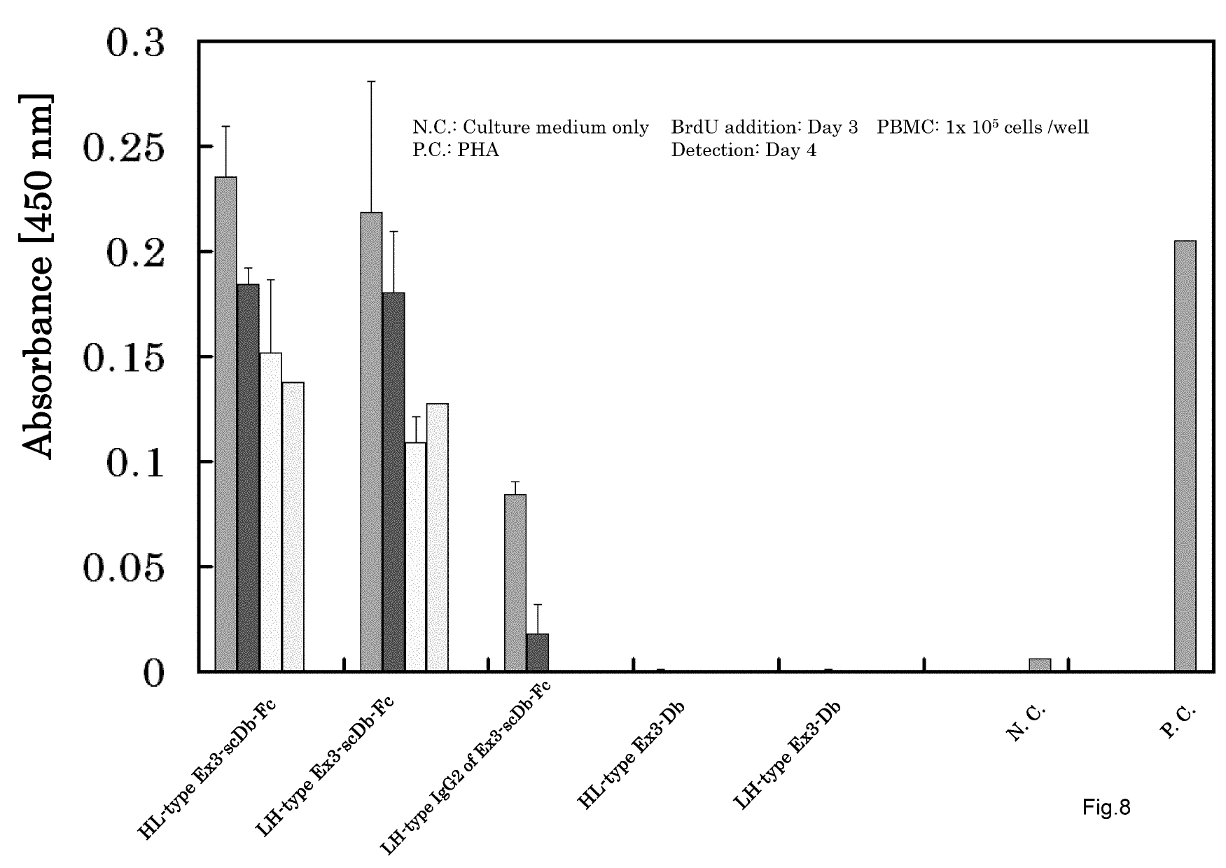

The FcR-binding capability was evaluated by PBMC proliferation assay with a method known for those skilled in the art, for example the method of Example 4 in Patent Document 2, using peripheral blood lymphocytes (PBMC). As a result, it was shown to have an extremely lower growth capability than HL-type or LH-type Ex3-scDb-Fc having the Fc region of IgG1 demonstrating that its FcR-binding capability had been reduced (FIG. 8). The LH-type or HL-type Ex3-scDb-Fc having the Fc region of IgG1 are the antibodies disclosed in Patent Documents 2 and 3, respectively.

(2) EGFR-Binding Kinetic Analysis by SPR

Binding kinetic analysis was carried out by SPR (J Biol Chem. 2010 July 2:285 (27): 20844-9) using EGFR (immobilized amount: 3690 RU) in order to evaluate the effects of structural change in the hinge or Fc region on the antigen-binding capability. The results showed that LH-type IgG2 of Ex3-scDb-Fc had the K_{D} equivalent to that of LH-type Ex3-scDb-Fc.

TABLE 2
Binding Evaluation by SPR

| | Kₐ(10⁵/Ms) | K_{d}(10⁻⁵/s) | K_{A}(10⁸/M) | K_{D}(10⁻⁹M) | Chi² |
|---|---|---|---|---|---|
| LH-type | 1.21 | 57.1 | 2.13 | 4.70 | 0.226 |
| Ex3-scDb-Fc | | | | | |
| LH-type IgG2 | 1.51 | 18.0 | 8.40 | 1.19 | 0.398 |
| of Ex3-scDb-Fc | | | | | |

(3) Evaluation of Cytotoxicity by MTS Assay

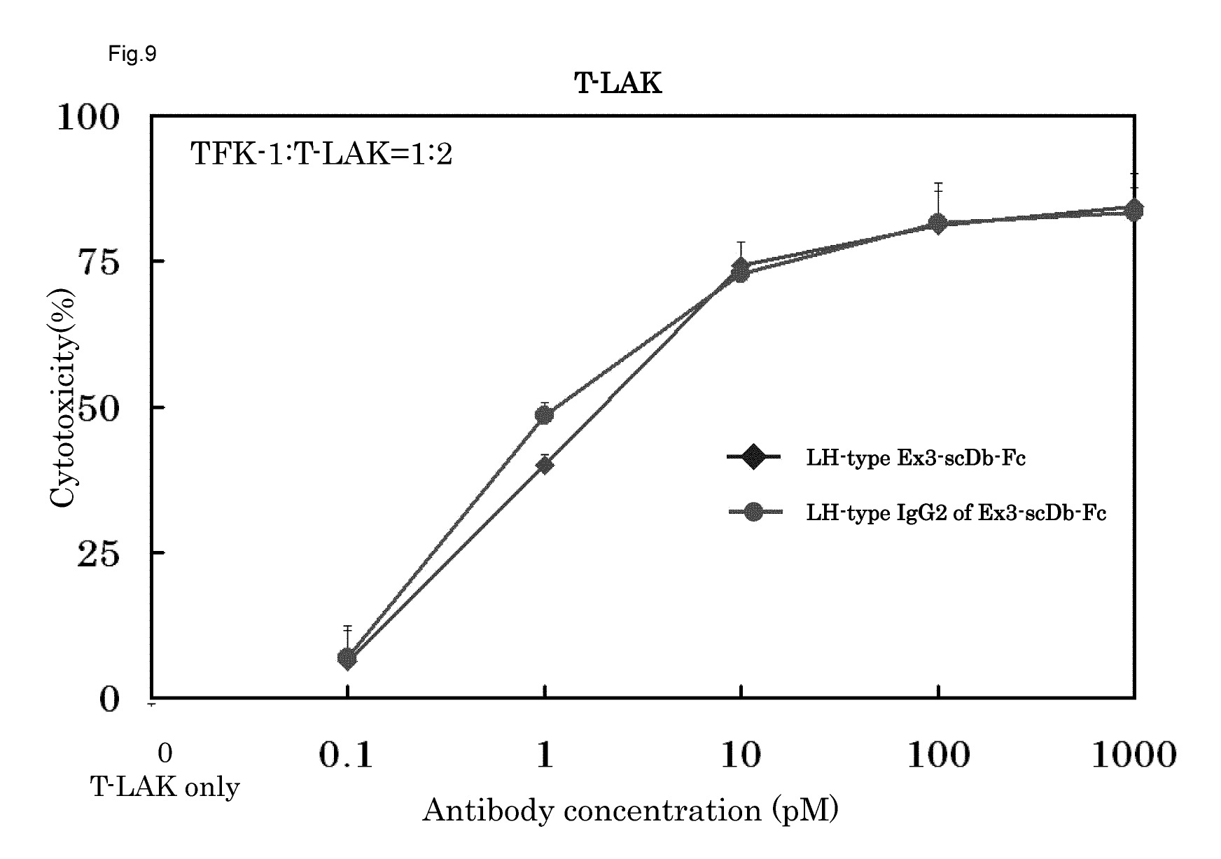

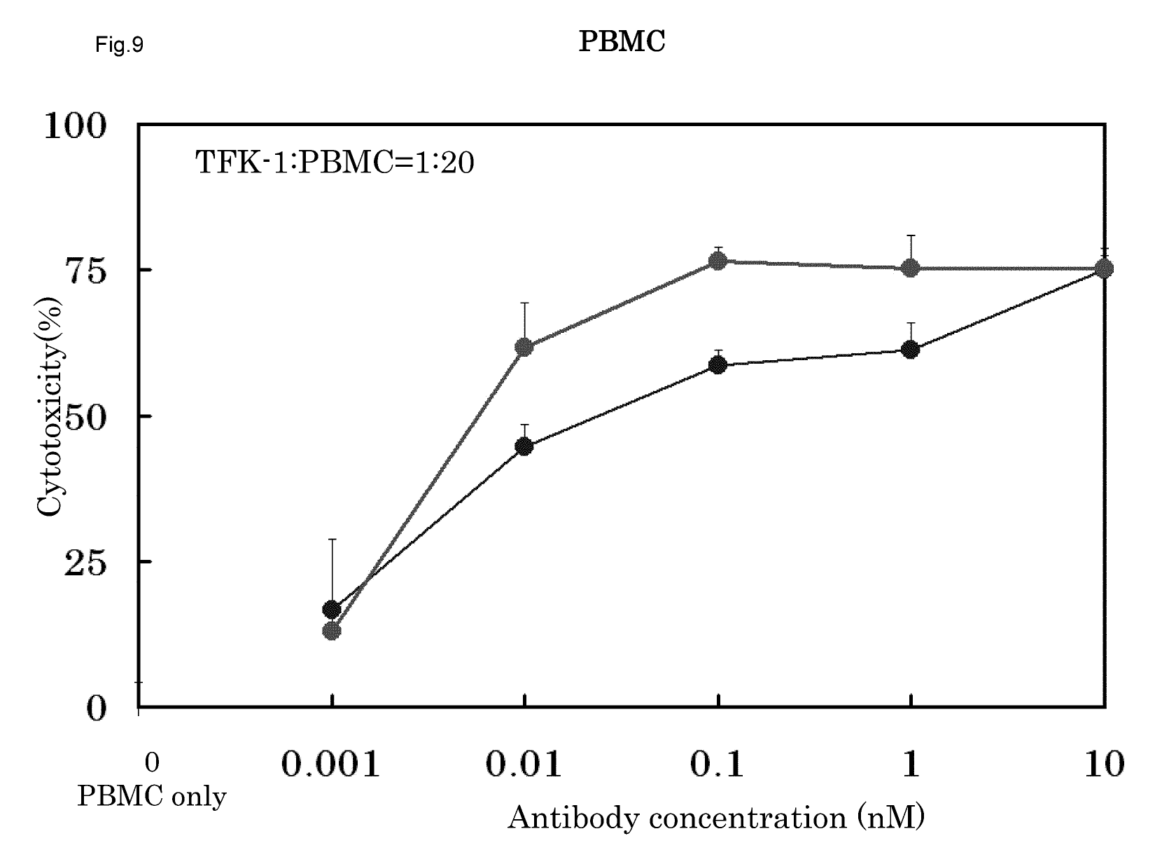

MTS assay for TFK-1 (Cell Resource Center for Biomedical Research, Institute of Development, Aging and Cancer, TOHOKU University, ID:TKG036) was carried out in accordance with a method known for those killed in the art, for example, the method of Example 5 in Patent Document 2, using T-LAK or PBMC as an effector cell in order to evaluate the effects of the above structural change on the cytotoxicity. The results showed that LH-type IgG2 of Ex3-scDb-Fc had the cytotoxicity for T-LAK equivalent to that of LH-type Ex3-scDb-Fc. On the other hand, the cytotoxicity of LH-type IgG2 of Ex3-scDb-Fc for PBMC was unexpectedly higher than that of LH-type Ex3-scDb-Fc (FIG. 9).

(4) Evaluation of the Capability of Inducing Cytokine Secretion

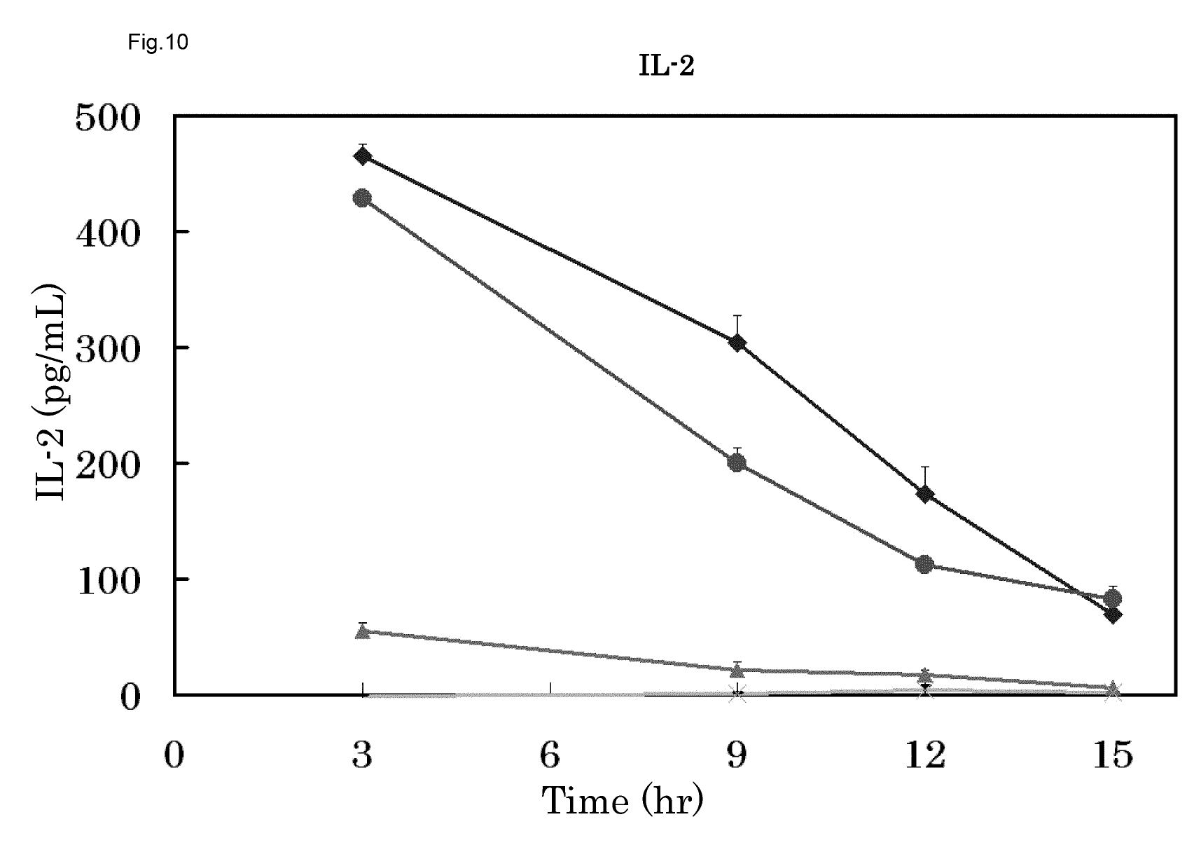

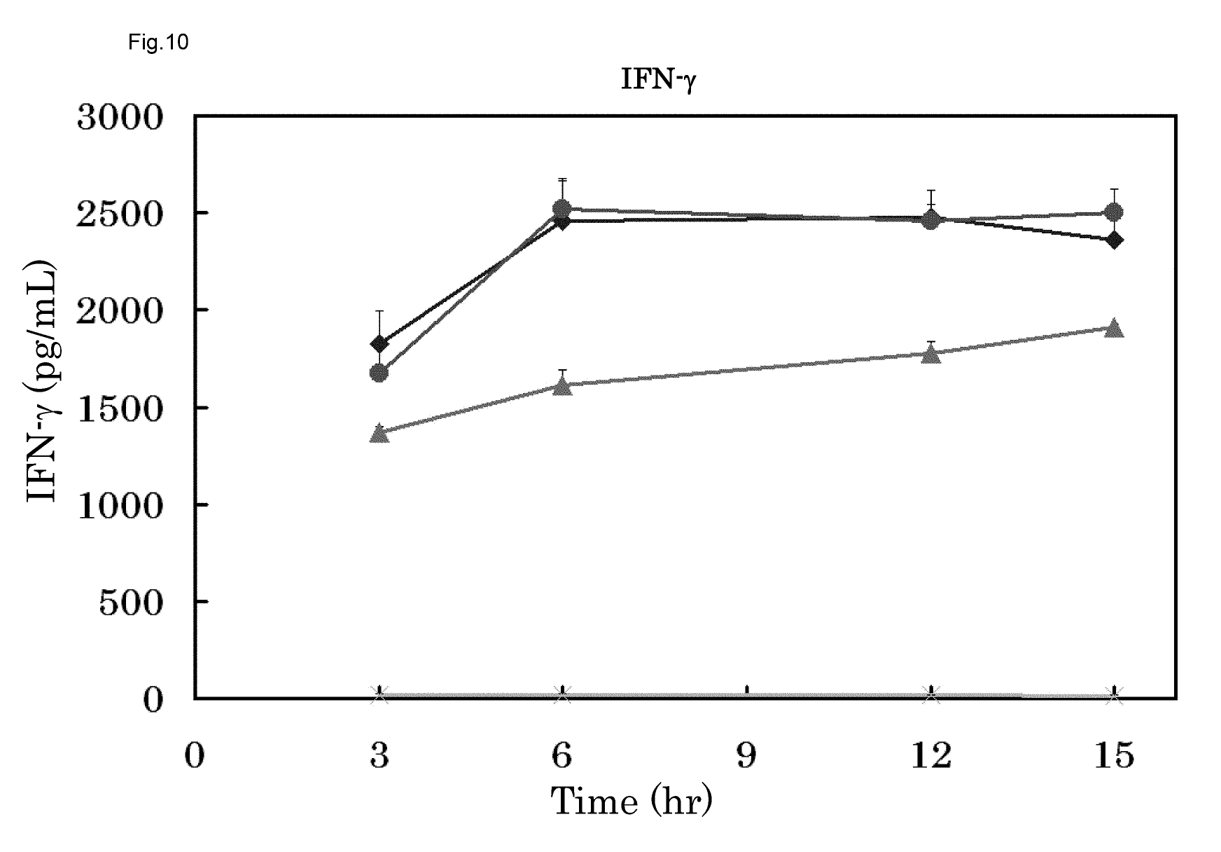

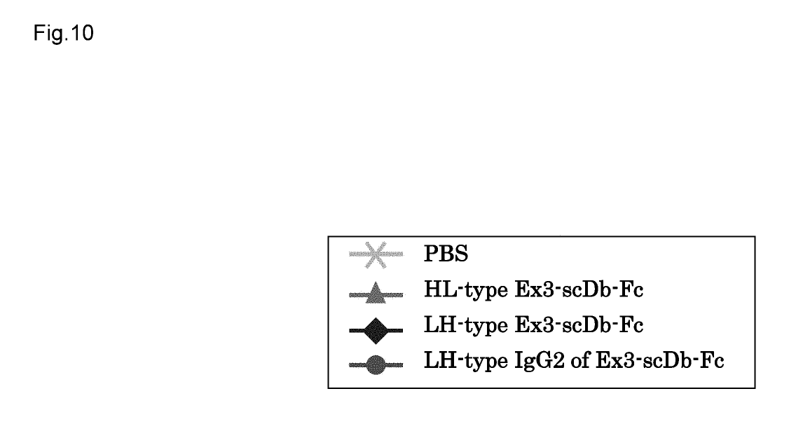

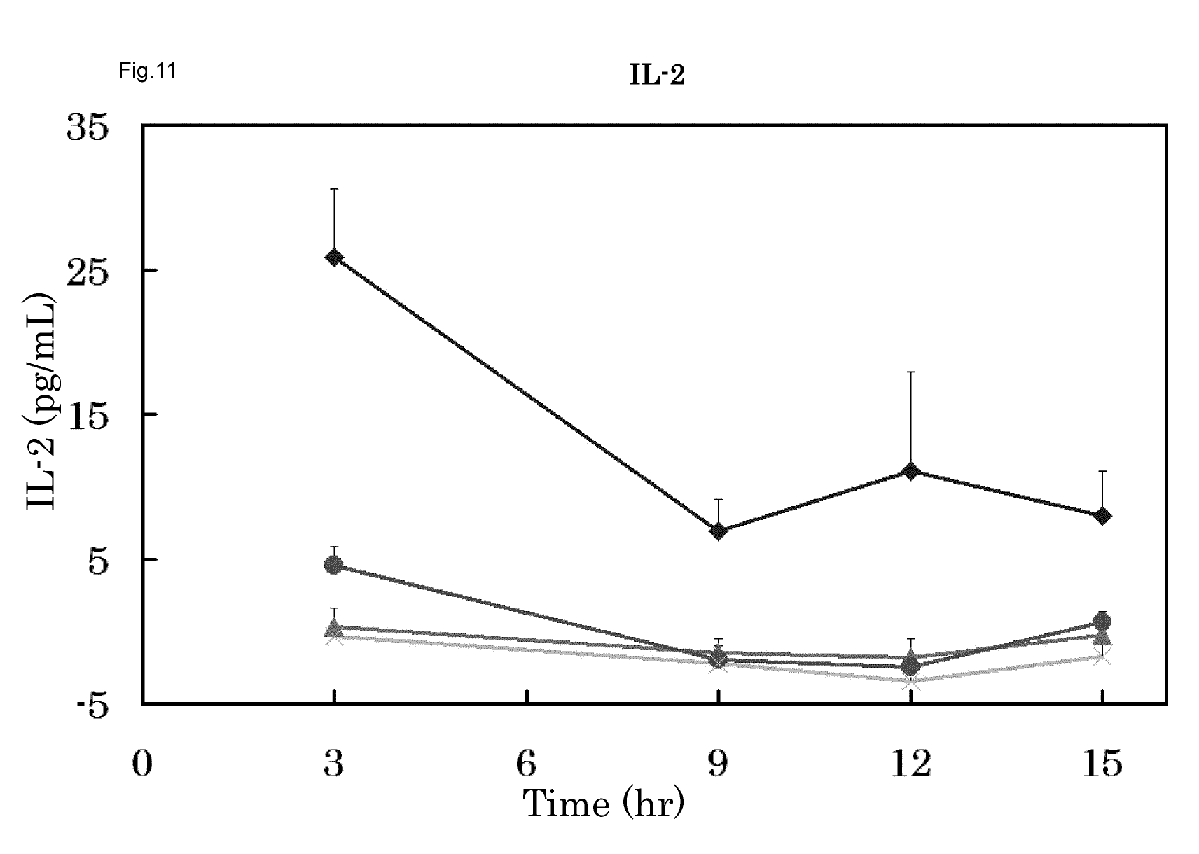

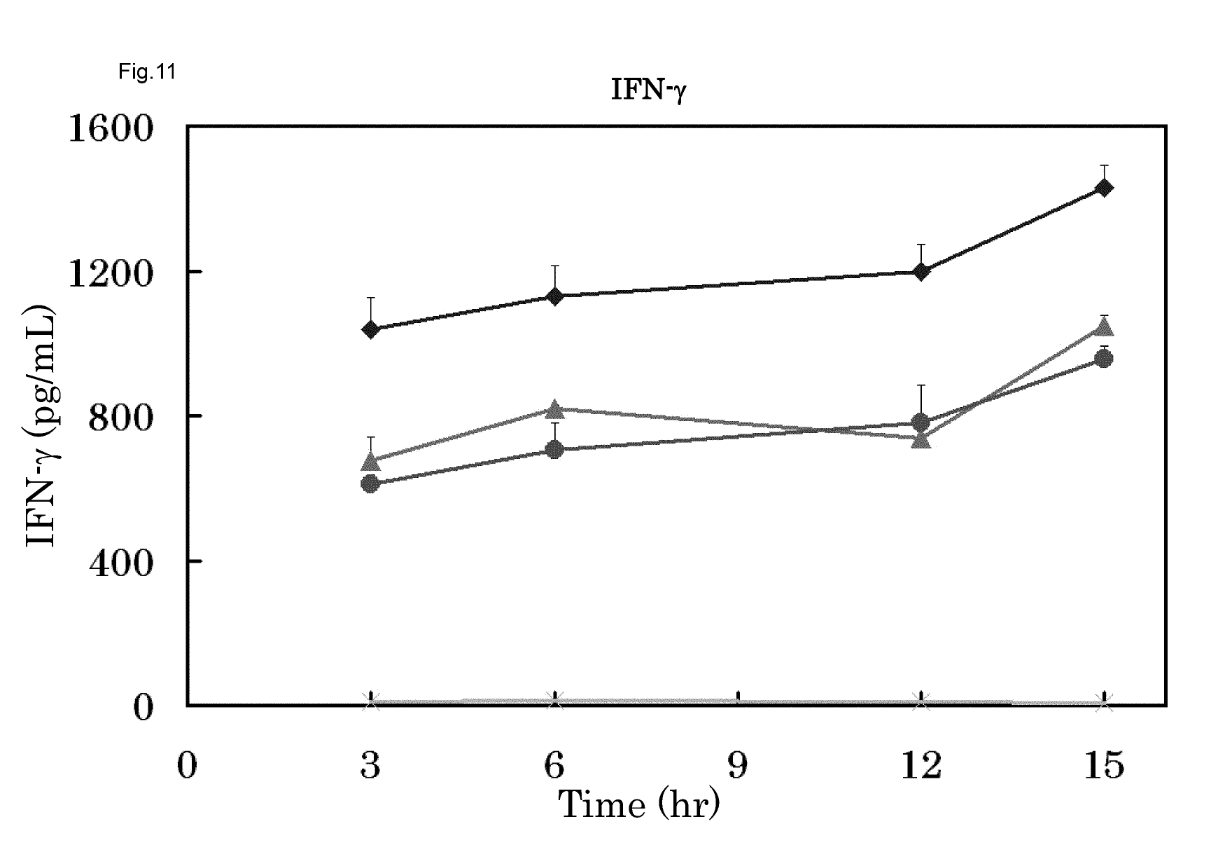

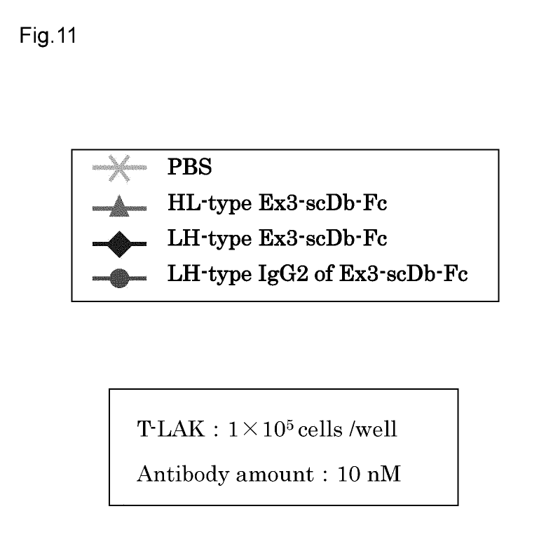

Time course of the capability of inducing secretion of IL-2 and IFN-γ was evaluated with respect to LH-type IgG2 of Ex3-scDb-Fc with the known method (J Biol Chem. 2011 January 21:286 (3): 1812-8) in the presence of both cancer cell and T-LAK, and of T-LAK only (FIG. 10 and FIG. 11).

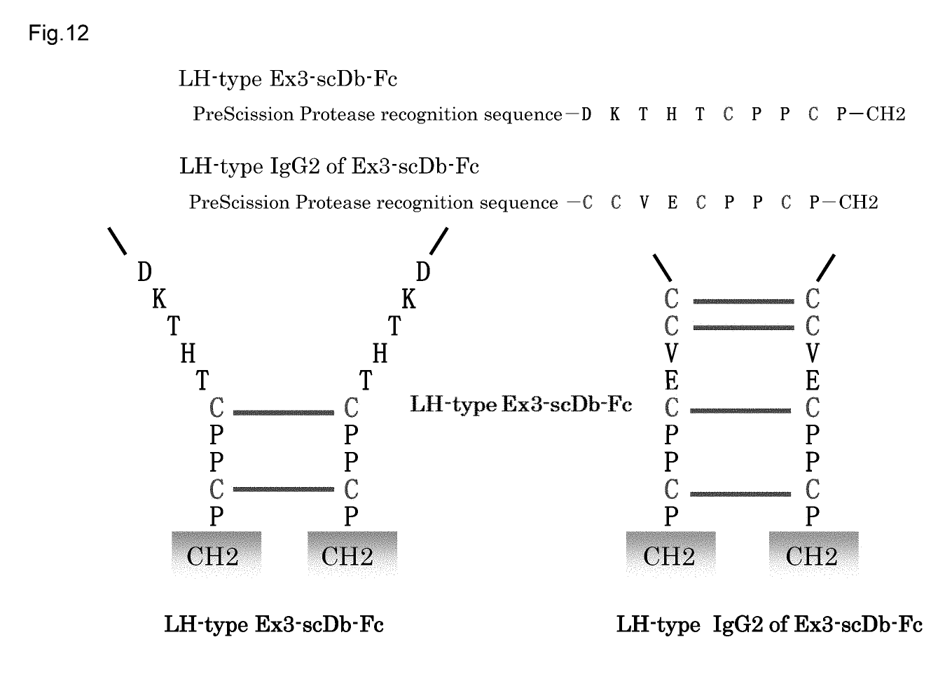

It was observed that it showed the inducing capability equivalent to that of LH-type Ex3-scDb-Fc in the presence of both the cancer cell and T-LAK. On the other hand, its inducing capability was lower than that of LH-type Ex3-scDb-Fc and equivalent to that of HL-type in the presence of T-LAK only. There are four cysteines in the hinge of LH-type IgG2, and a formation site of the disulfide binding is present directly after a PreScission Protease recognition sequence, as shown in FIG. 12. It is therefore considered that the flexibility of the hinge was reduced compared to LH-type Ex3-scDb-Fc wherein there are five residues between the PreScission Protease recognition sequence and the cysteine, so that the binding between OKT3 and CD3 was affected so as to reduce the T-LAK-activating capability. However, since cytotoxicity was not affected as shown in the previous Example, it is considered that the activating capability for T-LAK only will not make any serious effect on the cytotoxicity.

(5) Evaluation of T Cell-Activating Capability

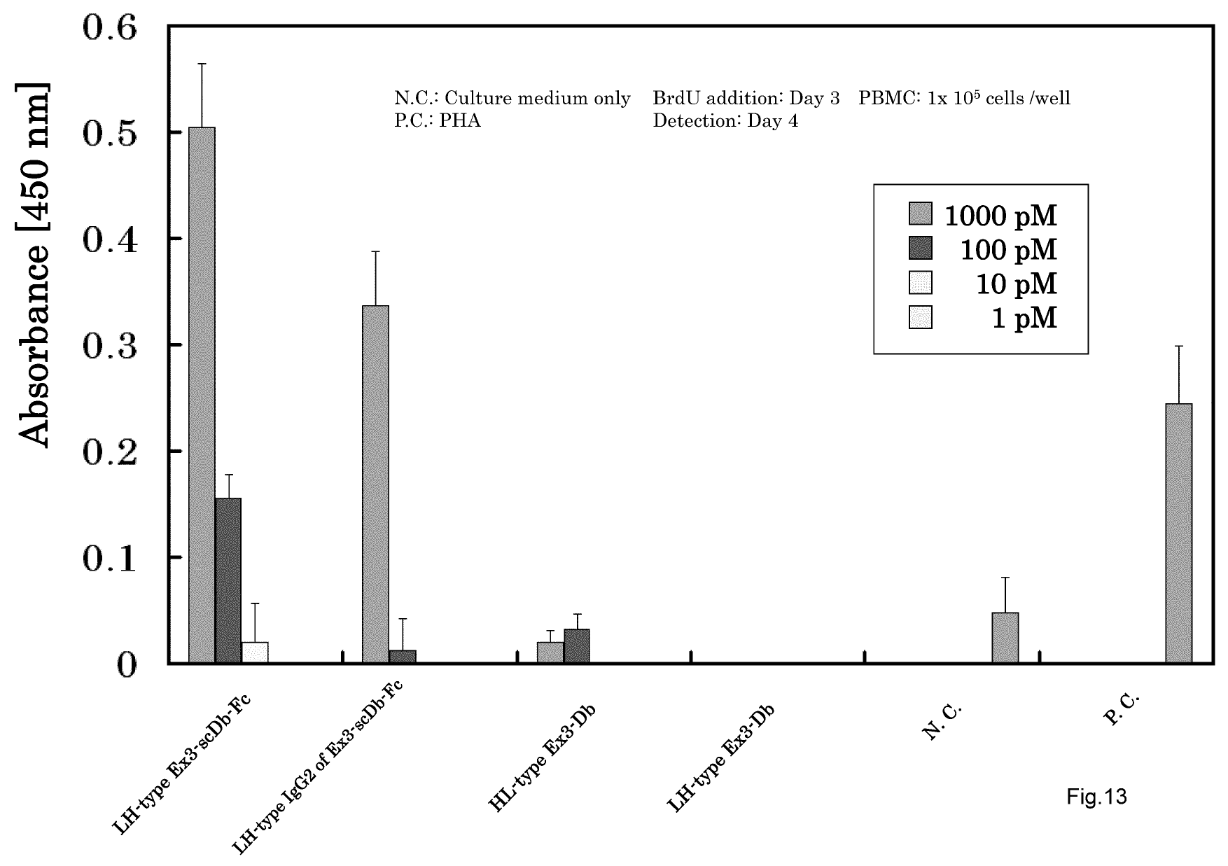

Since the T-LAK-activating capability of LH-type IgG2 of Ex3-scDb-Fc was reduced, it was thought that its T-LAK growth-inducing capability might be affected as well. Accordingly, the above proliferation assay was carried out using T-LAK (FIG. 13), confirming that the T-LAK growth-inducing capability was reduced when compared to that of LH-type Ex3-scDb-Fc.

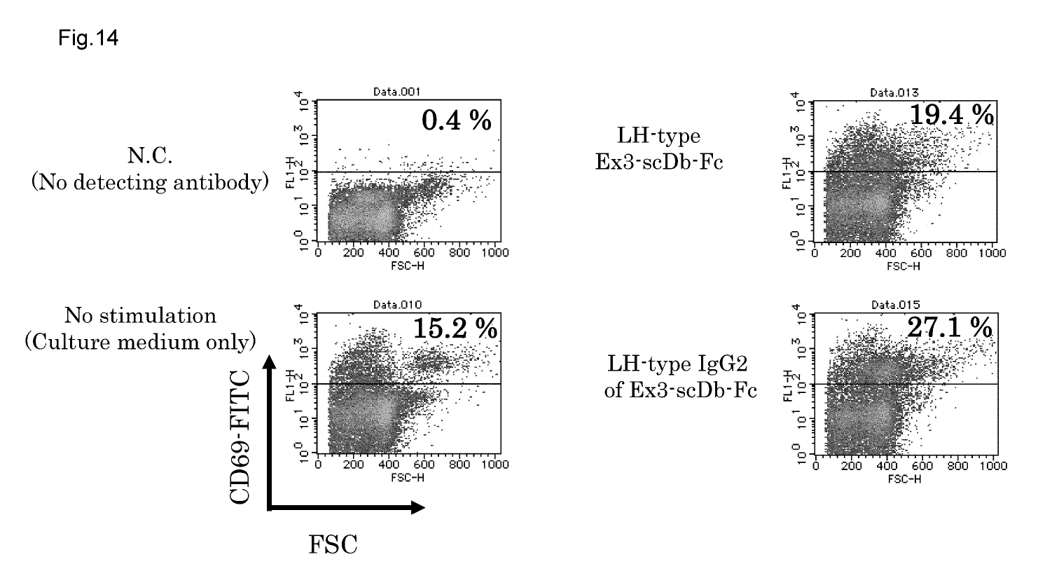

The expression of CD69 in PBMC after the addition of LH-type IgG2 of Ex3-scDb-Fc was evaluated by FCM (FIG. 14). It was shown that LH-type IgG2 of Ex3-scDb-Fc induced a higher expression of CD69 than LH-type Ex3-scDb-Fc. From these results, it was considered that the activating capability of LH-type IgG2 of Ex3-scDb-Fc was lower than LH-type Ex3-scDb-Fc due to the problem in its hinge when the T cell such as T-LAK was present alone. However, since the Fc region of the antibody according to the present invention is originated from IgG2 that is lower in the binding capability to FcR, LH-type IgG2 of Ex3-scDb-Fc could promote a higher activation in PBMC comprising FcR-positive effector cells such as NK cell as it bonded only to the T cell without being inhibited by the binding to the NK cell.

### EXAMPLE 4

Evaluation of Stability of LH-Type IgG2 of Ex3-scDb-Fc

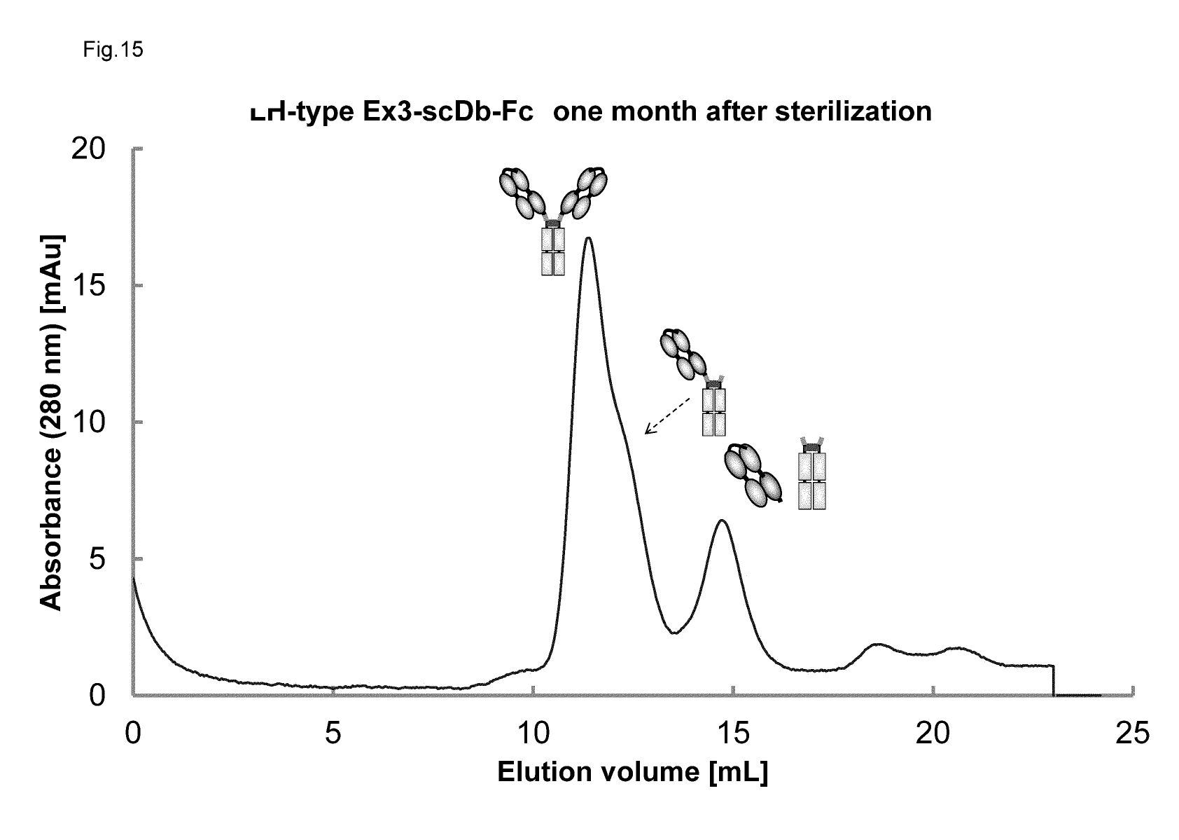

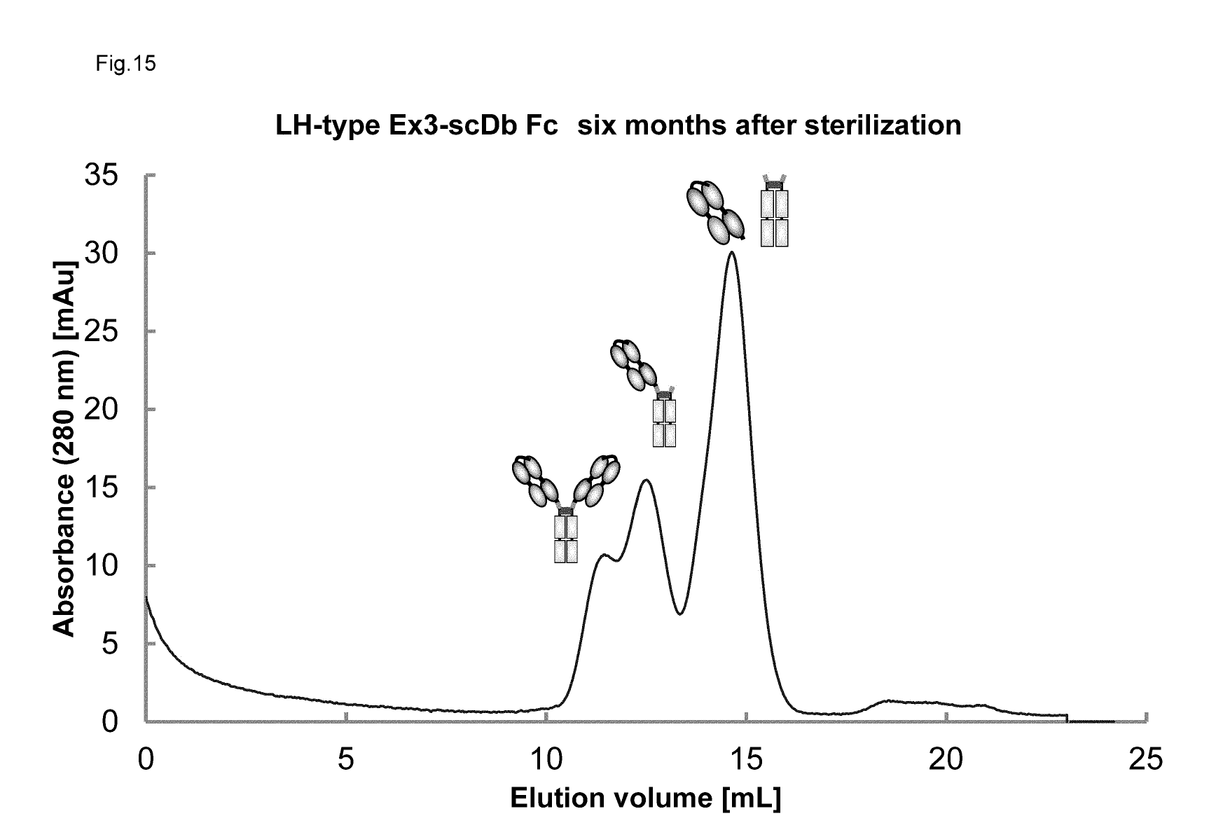

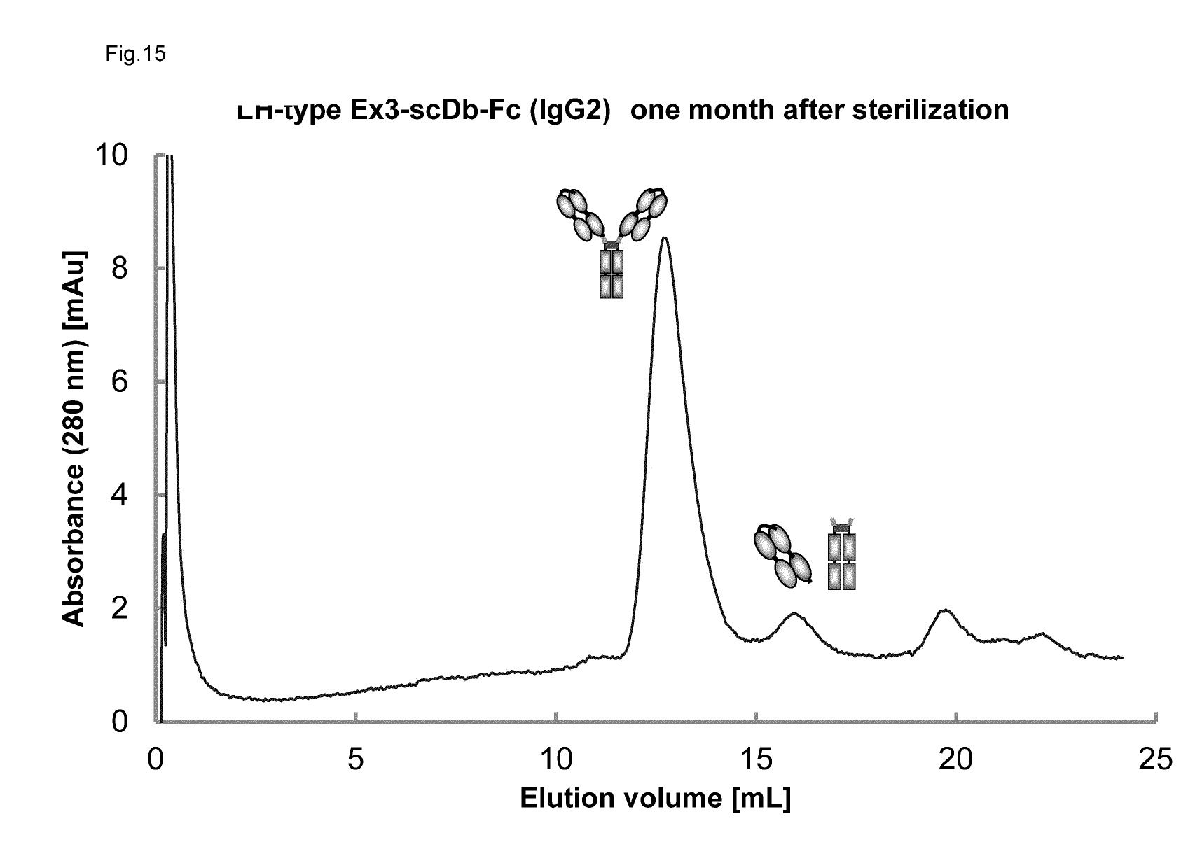

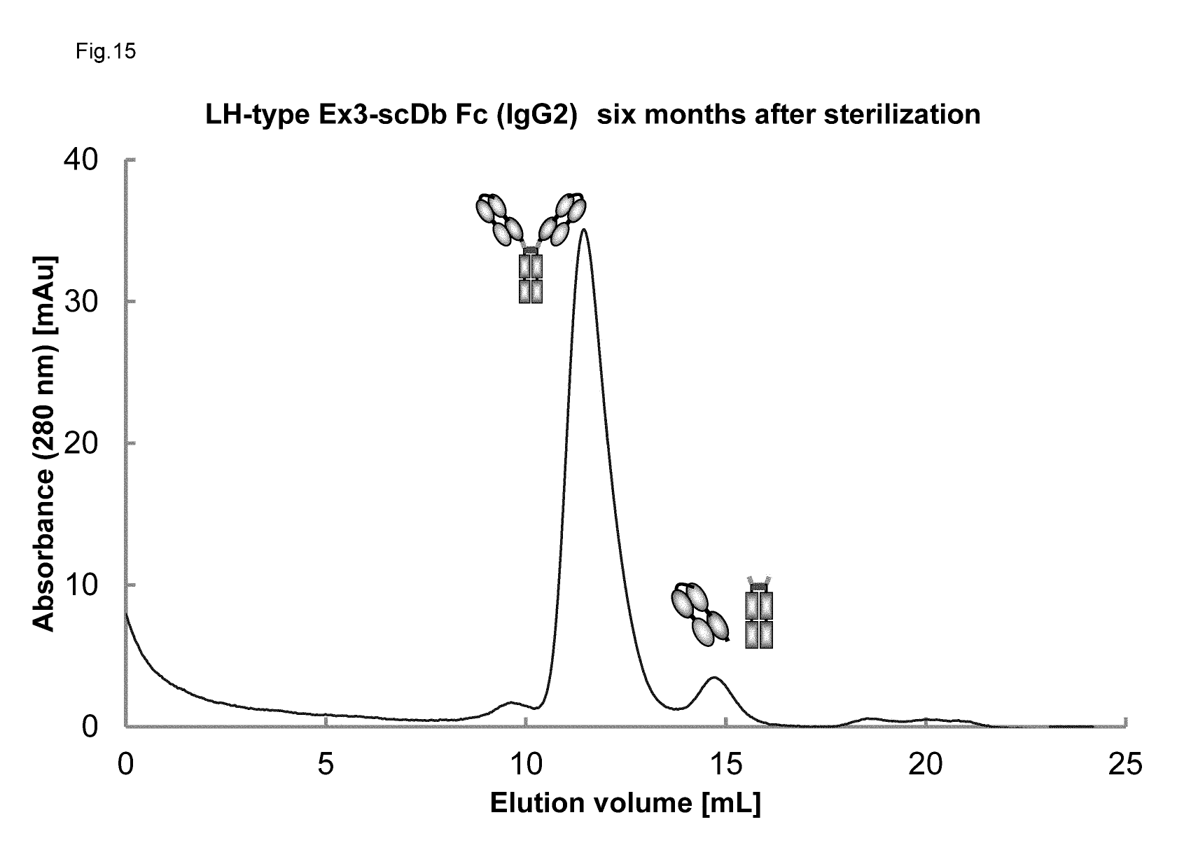

Ex3-scDb-Fc has the problem that it would be fragmented in the vicinity of the hinge region. In order to evaluate the stability of LH-type IgG2 of Ex3-scDb-Fc according to the present invention and LH-type IgG1 of Ex3-scDb-Fc (the antibody disclosed in Patent Document 3), each sample after the purification with the gel filtration chromatography was stored for a certain period of time and subjected again to the gel filtration chromatography to detect the occurrence of fragmentation. In order to exclude the effect by a factor such as protease secreted from microorganisms, the samples were subjected to filter-sterilization treatment before starting the above experiment. The results are shown in FIG. 15.

It was shown that while a lot of fragmentation of LH-type IgG1 of Ex3-scDb-Fc were observed after 6 months, little fragmentation was observed in LH-type IgG2 of Ex3-scDb-Fc after the lapse of such a long period of time, confirming that its fragmentation was significantly inhibited.

### EXAMPLE 5

In Vivo Test of LH-Type IgG2 of Ex3-scDb-Fc

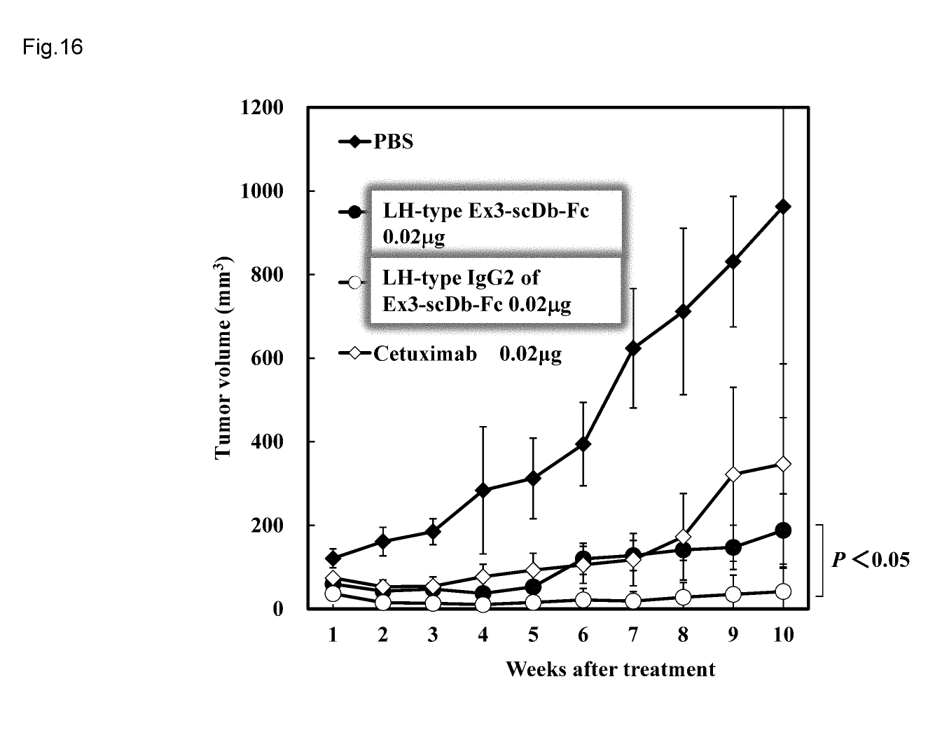

In vivo test of LH-type IgG2 of Ex3-scDb-Fc was carried out with the known method (Protein Eng Des Sel. 2013 May: 26(5): 359-67) using a tumor-carrying mouse (FIG. 16). After mixing TFK-1 (5×10⁶) and T-LAK (1×10⁷), the mixture was subcutaneously transplanted, and each sample was then administered one hour later.

As a result, LH-type Ex3-scDb-Fc showed a stronger tumor growth-inhibiting activity than that of a commercially available anti-EGFR antibody drug, Cetuximab, and LH-type IgG2 of Ex3-scDb-Fc showed a significantly stronger activity than LH-type IgG1 of Ex3-scDb-Fc.

### INDUSTRIAL APPLICABILITY

The present invention reduces the effector property so as to inhibit the side effects, avoids the steric hindrance, and improves the pharmaceutical effects of the various kinds of bispecific antibodies comprising Fc region by using a polypeptide derived from IgG2 as the Fc region. Furthermore, the present invention provides the technique to stably maintain the function of said antibodies for a long period of time. As a result, the present invention will accelerate and contribute a lot to the development of antibody drugs in the next generation.

1. A bispecific antibody for human EGF receptor 1 and CD3, comprising a variable region comprising a humanized variable region of the light chain (5L: SEQ ID NO:2) and a humanized variable region of the heavy chain (5H: SEQ ID NO:4) of an anti-human EGF receptor 1 antibody 528, and a humanized variable region of the light chain (OL: SEQ ID NO:6) and of a humanized variable region of the heavy chain (OH: SEQ ID NO:8) of an anti-CD3 antibody OKT; a hinge region; and an Fc region, characterized by that the Fc region is originated from human IgG2 sub-class.

2. The antibody according to claim 1, wherein the variable region of the light chain is located at an N-end side of the variable region of the heavy chain (LH-type) in each polypeptide.

3. The antibody according to claim 1 or 2, which has the same domain number as an IgG-type immunoglobulin.

4. The antibody according to claim 3, wherein a single polypeptide chain having the structure represented by (OL5H)-(peptide linker)-(5LOH) is bonded to the Fc region of a human antibody via a hinge region.

5. A single-chain polypeptide constituting the antibody of claim 1.

6. A nucleic acid molecule encoding the polypeptide of claim 5.

7. A replicable cloning vector or an expression vector containing the nucleic acid molecule of claim 6.

8. The vector of claim 7, which is a plasmid vector.

9. A host cell transformed with the vector of claim 7 or 8.

10. The hose cell of claim 9, which is a mammalian cell.

11. A method for the production of the antibody of claim 1, comprising culturing a host cell containing a replicable cloning vector or an expression vector containing a nucleic acid molecule encoding single-chain polypeptides constituting the antibody to express the nucleic acid molecule, and collecting and purifying the single-chain polypeptides constituting the antibody, assembling the resulting single-chain polypeptides, and separating and collecting the antibody thus formed.

12. A pharmaceutical composition comprising the antibody of claim 1 as an active ingredient.

13. The pharmaceutical composition of claim 12 for use in eliminating, hurting, damaging and/or reducing tumor cells.
